(19) United States
(12) Patent Application Publication
(10) Pub. No.: US 2013/0071375 A1
(43) Pub. Date: Mar. 21, 2013

(54) COMPOSITIONS AND METHODS FOR TREATING INFLAMMATION

(71) Applicants: Jong-Sup Bae, Daegu (KR); Alireza R. Rezaie, Eureka, MO (US)

(72) Inventors: Jong-Sup Bae, Daegu (KR); Alireza R. Rezaie, Eureka, MO (US)

(73) Assignee: SAINT LOUIS UNIVERSITY, St. Louis, MO (US)

(21) Appl. No.: 13/591,091

(22) Filed: Aug. 21, 2012

Related U.S. Application Data
(60) Provisional application No. 61/526,082, filed on Aug. 22, 2011.

(51) Int. Cl. C07K 19/00

(52) U.S. Cl. CPC C07K 19/00
USPC 424/94.64; 435/188

(57) ABSTRACT

The present invention provides methods for treating sepsis comprising administering to an individual an effective amount of a chimeric protein.

### CROSS-REFERENCES TO RELATED APPLICATIONS

The Present Application is based on and claims the benefit of priority from U.S. Provisional Patent Application Ser. No. 61/526,082, entitled “METHODS FOR TREATING INFLAMMATION” and filed on Aug. 22, 2011 with the United States Patent and Trademark Office, the contents of which are hereby incorporated by reference in their entirety to the extent permitted by law.

### STATEMENT OF GOVERNMENT SUPPORT

This work was funded in part by grant HL 101917 from the National Institutes of health. The United States Government may have certain rights in the invention.

### INTRODUCTION

The non-histone, chromatin-associated nuclear protein, high mobility group box 1 (HMGB1), has been identified as a potent pro-inflammatory extracellular cytokine and a late mediator of endotoxin lethality in mice [1,2], and is known to be secreted and/or released to plasma at high levels with late kinetics in severe sepsis and in experimental models of endotoxemia. [5] It can be secreted into intravascular spaces by cells of the innate immune system or passively released by damaged tissues and necrotic cells in response to bacterial endotoxin and/or trauma. [1-4] It has also been demonstrated that HMGB1 can be released from human endothelial cells in response to both endotoxin and TNF-α. [9-11]

Following its release to intravascular spaces, HMGB1 is known to interact with specific cell surface receptors to amplify inflammatory responses by inducing the expression of pro-inflammatory cytokines. [8-11] It is believed to trigger the activation of the endothelium and leukocytes by binding to at least three pathogen-associated cell surface pattern recognition receptors; toll-like receptors (TLR) [2 and 4] and the receptor for advanced glycation end products (RAGE), thereby inducing TNF-α expression and NF-κB activation in target cells. [6-9] Binding of HMGB1 to these receptors on endothelial cells induces the expression of adhesion molecules and stimulates the production of an array of pro-inflammatory cytokines that are involved in mediating leukocyte adherence, increased vascular permeability, coagulation activation and microvascular thrombosis. [9-11] A high concentration of HMGB1 in the plasma of patients with severe sepsis correlates with a poor prognosis and high mortality, and its pharmacological inhibition improves survival in animal models of acute inflammation and severe sepsis in response to endotoxin challenge. [5,12]

Activated protein C (APC) is a plasma serine protease that down-regulates thrombin generation by degrading the procoagulant cofactors Va and VIIIa by limited proteolysis. APC is generated when thrombin forms a complex with thrombomodulin on endothelial cell surface to activate the zymogen protein C [32]. The anticoagulant function of APC in degradation of both cofactors is stimulated by protein S. The importance of APC in regulation of blood coagulation can be illustrated by the observation that a heterozygous protein C deficiency is associated with high risk of venous thrombosis, and its homozygous deficiency causes purpura fulminans, which is fatal unless treated by protein C replacement therapy [33].

In addition to its anticoagulant role, APC also possesses antiinflammatory properties, which have led to it being the only currently approved drug for severe sepsis [13]. However, high concentrations are required which have a bleeding side effect in certain patients [13]. Results from several laboratories have demonstrated that APC elicits potent cytoprotective and antiinflammatory responses when it binds to endothelial protein C receptor (EPCR) to activate protease-activated receptor 1 (PAR-1) on endothelial cells. [14-18]. The EPCR and PAR-1 dependent antiinflammatory effect of APC is believed to be mediated through its ability to suppress the NF-κB dependent expression of pro-inflammatory cytokines and to inhibit the interaction and migration of leukocytes across the endothelium. [19, 20] APC also inhibits apoptosis and protects the endothelium from the hyper-permeability effect of inflammatory mediators. [20-22]

The EPCR and PAR-1 dependent cytoprotective and antiinflammatory activities of APC have been confirmed in several acute animal models of inflammation and severe sepsis [14-18]. In addition to APC, thrombin also activates PAR-1. The activity of thrombin toward PAR-1 is three orders of magnitude higher than that of APC. Interestingly, it has been reported that when thrombin activates PAR-1, it elicits a pro-inflammatory response. The basis for the paradoxical effect of PAR-1 activation by either APC or thrombin is not known. However, noting that the activity of thrombin toward PAR-1 is very high, one hypothesis is that the dose of receptor cleavage by two proteases may be critical for the specificity of PAR-1 signaling. [20,22, 34] Thrombin is the only known physiologic activator of protein C, and it can be produced at a concentration of much higher than APC. How APC can then activate PAR-1 in the presence of thrombin is not known.

To investigate whether the level of receptor activation by thrombin and APC determines the type of response in endothelial cells, a chimeric meizothrombin “PCgla/meizothrombin” (PCgla/MeizoTh) was constructed in which the γ-carboxyglutamic acid (Gla) domain of meizothrombin was substituted with the corresponding domain of APC (see FIG. 1 for the construction of PCgla/MeizoTh). [26, 35] This meizothrombin derivative retained its high specific activity toward PAR-1, interacted with EPCR with normal affinity, and was found to cleave PAR-1 at a rate approximately 1000 times higher than APC. [26] Unlike thrombin, however, the rapid cleavage of PAR-1 by PCgla/meizothrombin elicited a protective response in endothelial cells in response to pro-inflammatory mediators including LPS and TNF-α, suggesting that the binding of the Gla-domain of APC to EPCR determines the type of signaling response [20,26].

A potent protective activity for APC has been observed in an LPS-induced murine model of endotoxemia, which appeared to be mediated through APC proteolytically degrading intravascularly released nuclear histones independent of its interaction with EPCR and PAR-1. [24] A similar protective effect has been observed for the thrombin-thrombomodulin (TM) complex through the proteolytic degradation and inhibition of HMGB1. [25] However, the effect of APC on HMGB1 release and/or HMGB1 signaling has never been investigated.

### BRIEF DESCRIPTION OF THE FIGURES

FIG. 1. Scheme of the construction of the PCgla-containing mutant of prothrombin and its activation by Factor Xa to active PCgla/MeizoTh. (A) The proteolytic cleavage of wild-type prothrombin at its Arg-320 by Factor Xa yields an activation intermediate that is an active product called meizothrombin. A second cleavage at Arg-271 by Factor Xa is required to separate the catalytic domain of prothrombin from its non-catalytic domains (Gla, Kringle-1 and Kringle-2 domains) to yield thrombin. Further cleavages can occur at Arg-155 and Arg-284 in a feed-back reaction by both thrombin and meizothrombin. (B) The substitution of the Gla domain of prothrombin with the corresponding domain of protein C and of Arg-155, Arg-271 and Arg-284 residues of prothrombin with Alanines yields a mutant of prothrombin (PCgla/prothrombin-3A) which can be activated by Factor Xa through the cleavage of Arg-320 to yield PCgla/MeizoTh which cannot be further processed by either Factor Xa or the resulting mutant meizothrombin.

FIG. 2. Effect of APC on the LPS-mediated release of HMGB1. (A) HUVECs were stimulated with indicated concentrations of LPS for 16 hours and the release of HMGB1 was measured by an ELISA as described under “Materials and Methods”. (B) The LPS (100 ng/mL)-mediated HMGB1 release by HUVECs was monitored after treating the cell monolayer with indicated concentrations of APC for 3 hours. (C) The same as (B) except that cells were incubated with increasing concentrations of meizothrombin (MeizoTh) (white bars) or PCgla/MeizoTh (Black bars). (D) The same as (B or C) except that cells were pre-incubated with function-blocking antibodies to PAR-1 or EPCR (25 μg/mL for 30 min) before treating cells with each protease (100 nM APC, 2 nM PCgla/MeizoTh). All results are shown as means±SD (Standard Deviation) of five different experiments. *p<0.05 and **p<0.01 as compared to 0 (A) or LPS (B, C, and D).

FIG. 3. Effect of APC on the HMGB1-mediated expression of cell adhesion molecules in HUVECs. Confluent HUVECs were incubated with HMGB1 (1 μg/mL, for 16 h) after treating cells with indicated concentrations of APC for 3 h. The cell surface expression of VCAM-1 (A), ICAM-1 (B) and E-selectin (C) on HUVECs was measured by a cell-based ELISA as described under “Materials and Methods”. All results are shown as means±SD of five different experiments. *p<0.05 and **p<0.01 as compared to HMGB1.

FIG. 4. Analysis of the HMGB1-mediated THP-1 adhesion and migration in HUVECs. (A) Confluent HUVECs were incubated with HMGB1 (1 μg/mL, for 16 h) after treating cells with indicated concentrations of APC for 3 h and the THP-1 adherence to HUVECs was monitored as described under “Materials and Methods”. (B) The HMGB1 (1 μg/mL, for 16 h)-mediated migration of THP-1 across HUVEC cell monolayers was analyzed after treating cells with indicated concentrations of APC. (C and D) The same as (A and B) except that MeizoTh (white bars) or PCgla/MeizoTh (Black bars) were used to treat cells for 3 h prior to stimulation by LPS. All results are shown as means±SD of five different experiments. *p<0.05 and **p<0.01 as compared to HMGB1.

FIG. 5. Effect of PCgla/MeizoTh on the HMGB1-mediated expression of cell adhesion molecules in HUVECs. Confluent HUVECs were incubated with HMGB1 (1 μg/mL, for 16 hours) after treating the cells with indicated concentrations of PCgla/MeizoTh for 3 hours. The cell surface expression of VCAM-1 (A), ICAM-1 (B) and E-selectin (C) on HUVECs was measured by a cell-based ELISA as described under “Materials and Methods”. All results are shown as means±SD of five different experiments. *p<0.05 and **p<0.01 as compared to HMGB1.

FIG. 6. Effect of APC or PCgla/MeizoTh on the HMGB1-mediated NF-κB activation and TNF-α expression. Confluent HUVECs were incubated with HMGB1 (1 μg/mL, for 16 hours) after treating cells with APC (100 nM) for 3 hours. The activation of NF-κB (A) or the induction of TNF-α (B) in HUVECs was analyzed as described under “Materials and Methods.” In the presence of antibodies, cells were first pre-incubated with function-blocking antibodies to PAR-1 or EPCR (25 μg/mL for 30 minutes) before treating cells with APC. (C and D) The same as (A and B) except that instead of 100 nM APC, 2 nM MeizoTh (white bars) or 2 nM PCgla/MeizoTh (black bars) were used in the experiments. All results are shown as means±SD of five different experiments. **p<0.01 as compared to HMGB1.

FIG. 7. The Effect of siRNA knockdown of pattern recognition receptors on the HMGB1-mediated NF-κB activation and TNF-α expression in HUVECs. Confluent HUVECs were transfected with the control siRNA (1 μg for 3 days) or siRNA (1 μg for 3 days) specific for TLR2, TLR4 and RAGE individually or in combination of three before incubating cells with HMGB1 (1 μg/mL, for 16 h). The activation of NF-κB (A) or the induction of TNF-α (B) in HUVECs was analyzed as described under “Materials and Methods.”**p<0.01 as compared to HMGB1. ^{#}p<0.05 as compared to TLR2; ^{#}p<0.02 as compared to TLR4; ⁺p<0.05 as compared to RAGE; and ⁺p<0.02 as compared to either TLR2 or TLR4.

FIG. 8. Effect of APC on the HMGB1-mediated expression of pattern recognition receptors on HUVECs and the proteolytic cleavage of HMGB1 by PCgla/MeizoTh. (A) Confluent HUVECs were incubated with HMGB1 (1 μg/mL, for 16 hours) with or without pre-treating cells with protein C (100 nM), APC (100 nM), MeizoTh (2 nM) and PCgla/MeizoTh (2 nM) for 3 hours as described under “Materials and Methods.” The expression of TLR2 (white bars), TLR4 (grey bars) and RAGE (black bars) on HUVECs was measured by a cell-based ELISA as described under “Materials and Methods.” All results are shown as means±SD of five different experiments. **p<0.01 as compared to HMGB1. (B) The cleavage of HMGB1 by PCgla/MeizoTh was monitored in the absence and presence of TM by SDS-PAGE (10% under reducing conditions) followed by immunoblotting as described under “Materials and Methods.”

FIG. 9. Amino acid sequence for human prepro prothrombin, including the polypeptide of SEQ ID NO: 1 and showing the location of the 13 introns (A through M). The prepro leader sequence (numbered −43 to −1) is removed during biosynthesis by signal peptidase and a processing protease that hydrolyzes the R-A bond between −1 and 1, thereby releasing wild-type human prothrombin. The Gla-domain and the Kringle-1 and Kringle-2 domains are located within residues 1 through 271 of prothrombin, which constitute fragment 1. This fragment is released from prothrombin during its conversion to thrombin by Factor Xa. The light chain in thrombin is generated by the cleavage of the R319-I bond (shown as residue R49 in chymotrypsin numbering system in FIG. 1) by Factor Xa which also activates prothrombin to thrombin, and this chain is attached to the catalytic domain by a single disulfide bond. The serine protease or catalytic domain of thrombin contains 259 residues, including the three principal amino acids participating in the catalysis. These three amino acids (H363, D419, and S525) are circled. Three potential carbohydrate binding sites are shown by solid diamonds. The proposed disulfide bonds in human prothrombin have been placed by analogy to those in the bovine molecule. The single-letter code for amino acids in FIG. 9 are as follows: A, Alanine; R, Arginine; N, Asparagine; D, Aspartic acid; C, Cysteine; Q, Glutamine; E, Glutamic acid; G, Glycine; H, Histidine; I, Isoleucine; L, Leucine; K, Lysine; M, Methionine; F, Phenylalanine; P, Proline; S, Serine; T, Threonine; W, Tryptophan; Y, Tyrosine; V, Valine; γ, γ-carboxyglutamic acid.

### DEFINITIONS

When introducing elements of aspects of the invention or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements. The word “or” means any one member of a particular list and also includes any combination of members of that list, unless otherwise specified.

The term “about” is used herein to mean approximately, roughly, around, or in the region of. When the term “about” is used in conjunction with a numerical range, it modifies that range by extending the boundaries above and below the numerical values set forth. Preferably, the term “about” is used herein to modify a numerical value above and below the stated value by a variance of 20 percent up or down (higher or lower).

The term “polynucleotide” as used herein refers to a naturally occurring or synthetic polynucleotide, whether DNA or RNA or DNA-RNA hybrid, single-stranded or double-stranded, sense or antisense, which is capable of hybridization to a complementary nucleic acid by Watson-Crick base-pairing. Polynucleotides can also include nucleotide analogs (e.g., BrdU), and non-phosphodiester internucleoside linkages (e.g., peptide nucleic acid (PNA) or thiodiester linkages). In particular, polynucleotides can include, without limitation, DNA, RNA, cDNA, gDNA, ssDNA or dsDNA or any combination thereof.

As intended herein, the term “infection” is the colonization of a host organism by parasite species. Infecting parasites seek to use the host's resources to reproduce, often resulting in disease. Infections are usually considered to be caused by microscopic organisms or microparasites like viruses, prions, bacteria, and viroids, though larger organisms like macroparasites and fungi can also infect.

As intended herein, the term “sepsis” is a systemic inflammatory response syndrome in response to a confirmed infection, such as bacterial infection. When sepsis is associated with organ dysfunction, hypoperfusion, or hypotension, it is known as “severe sepsis.”

As used herein, “treatment” is an approach for obtaining beneficial or desired clinical results. For purposes of this invention, beneficial or desired clinical results include, but are not limited to, one or more of lessening severity, alleviation of one or more symptoms associated with sepsis. For instance, when an individual is affected with severe sepsis, “treatment” includes preventing/reducing the extent of sepsis and attenuating proinflammatory responses, for example by attenuating HMGB1-mediated pro-inflammatory signaling responses.

For the purpose of the present disclosure, an “effective amount” of drug, compound, or pharmaceutical composition is an amount sufficient to affect beneficial or desired clinical results in the treatment of sepsis. An effective amount can be administered in one or more administrations. As is understood in the clinical context, an effective amount of a drug, compound, or pharmaceutical composition may or may not be achieved when administered in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “individual” is a vertebrate, preferably a mammal, more preferably a human. Mammals include, but are not limited to, farm animals (such as cows), sport animals, pets (such as cats, dogs and horses), primates, mice and rats.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

As used herein, a “thrombin polypeptide” refers to any thrombin polypeptide (protein) including, but not limited to, recombinantly-produced polypeptide, synthetically-produced polypeptide and thrombin extracted from cells. Thrombin polypeptides include precursor thrombin polypeptides having signal sequences and mature thrombin polypeptide. Thrombin polypeptides include related polypeptides from different species including, but not limited to animals of human and nonhuman origin. Thrombin polypeptide amino acid sequences can contain varying number of amino acid residues. For example, the human thrombin polypeptide of SEQ ID NO:1 is known to include 622 amino acids. Other polypeptides also can be shorter than 622 amino acids, provided that a polypeptide retains an activity of the thrombin. Human thrombins include allelic variant isoforms among individuals, alternative splice variants, synthetic molecules from nucleic acids, protein isolated from human tissue and cells, chimeric proteins including a thrombin polypeptide, and modified forms thereof.

As used herein, an “activity” or “property” of a polypeptide (protein) refers to any activity or property exhibited by a protein that can be assessed. Such activities include those observed or exhibited in vitro or in vivo (typically referred to as a biological activity). These activities include, but are not limited to, the treatment of inflammation and the treatment of sepsis.

As used herein, a “fragment of a given polypeptide” refers to any fragment that exhibits one or more biological activities of the full-length polypeptide.

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, non-naturally occurring amino acids), as well as other modifications known in the art.

As used herein, “unmodified polypeptide,” “unmodified protein,” “unmodified thrombin,” “unmodified thrombin polypeptide,” or grammatical variations thereof, refer to a starting polypeptide (protein) that is selected for modification. The starting unmodified target polypeptide can be the naturally occurring, wild type (WT) form of a protein. In addition, the starting unmodified polypeptides previously can have been altered or mutated, such that they differ from the native wild-type isoform, but are nonetheless referred to herein as starting unmodified polypeptides relative to the subsequently modified polypeptides disclosed herein. Thus, existing polypeptides known in the art that have previously been modified to have a desired increase or decrease in a particular activity compared to an unmodified reference protein can be selected and used herein as the starting “unmodified protein.” For example, a polypeptide that has been modified from its native form by one or more single amino acid changes and possesses either an increase or decrease in a desired activity, such as anti-sepsis therapeutic activity, can be utilized with the methods provided herein as the starting unmodified polypeptide for further modification of either the same or a different activity.

Likewise, existing polypeptides known in the art that previously have been modified to have a desired alteration, such as an increase or decrease, in a particular activity compared to an unmodified or reference protein can be selected and used as provided herein for identification of structurally homologous loci on other structurally homologous polypeptides. For example, a polypeptide that has been modified by one or more single amino acid changes and possesses either an increase or decrease in a desired activity (e.g., treatment of sepsis) can be utilized with the methods provided herein to identify structurally homologous polypeptides, corresponding structurally homologous loci that can be replaced with suitable replacing amino acids and tested for either an increase or decrease in a desired or selected activity.

As intended herein, “modified polypeptide,” “modified protein,” “modified thrombin,” “modified chimeric polypeptide,” “derivative,” or grammatical variations thereof, refer to derivatives of a protein which may be obtained, for example, by subjecting an unmodified protein, for example thrombin, to one or more modifications. Example modifications include mutations, truncations, enzymatic digestions, formation of chimeric proteins with fragments of other proteins, and/or changing its post-translational modifications. Mutations may be one or more amino acid replacements, insertions, deletions and/or any combination thereof.

As used herein, “in a position or positions corresponding to an amino acid position” of a protein refers to amino acid positions that are determined to correspond to one another based on sequence and/or structural alignments with a specified reference protein. For example, a position corresponding to an amino acid position of human thrombin set forth as SEQ ID NO: 1 can be determined empirically by aligning the sequence of amino acids set forth in SEQ ID NO: 1 with a particular polypeptide of interest. Corresponding positions can be determined by such alignment by one of skill in the art using manual alignments or by using the numerous alignment programs available (for example, BLASTP). Corresponding positions also can be based on structural alignments, for example, by using computer simulated alignments of protein structure. Recitation that amino acids of a polypeptide correspond to amino acids in a disclosed sequence refers to amino acids identified upon alignment of the polypeptide with the disclosed sequence to maximize identity or homology (where conserved amino acids are aligned) using a standard alignment algorithm, such as the GAP algorithm. As used herein, “at a position corresponding to” refers to a position of interest (e.g., base number or residue number) in a nucleic acid molecule or protein relative to the position in another reference nucleic acid molecule or protein. The position of interest to the position in another reference protein can be in, for example, a precursor protein, an allelic variant, a heterologous protein, an amino acid sequence from the same protein of another species, and the like. Corresponding positions can be determined by comparing and aligning sequences to maximize the number of matching nucleotides or residues. For example, identity between the sequences can be greater than 95%, greater than 96%, greater than 97%, greater than 98% and more particularly greater than 99%. The position of interest is then given the number assigned in the reference nucleic acid molecule or polypeptide sequence. One of skill in the art would understand that for a modified thrombin polypeptide compared to an unmodified thrombin polypeptide, amino acid residue 1 of the modified polypeptide corresponds to amino acid residue 1 of the unmodified thrombin polypeptide. One of skill in the art would also understand that for a modified precursor thrombin polypeptide compared to a precursor unmodified thrombin polypeptide, amino acid residue 1 of the modified polypeptide corresponds to amino acid residue 1 of the unmodified thrombin polypeptide.

As used herein, the term “nucleic acid molecule” encompasses both deoxyribonucleotides and ribonucleotides and refers to a polymeric form of nucleotides including two or more nucleotide monomers. The nucleotides can be naturally occurring, artificial (such as PNA and XNA), modified, and unusual nucleotides such as those referred to in 37 C.F.R. §§1.821-1.822. Examples of nucleic acid molecules include oligonucleotides that typically range in length from 2 nucleotides to about 100 nucleotides, and polynucleotides, which typically have a length greater than about 100 nucleotides.

As used herein, the terms “homology” and “identity” are used interchangeably but homology for proteins can include conservative amino acid changes. Usually, to identify corresponding positions the sequences of amino acids are aligned so that the highest order match is obtained (see, for example: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griflin, A. M., and Griflin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. SIAM. I. Applied Math 48: 1073 (1988)).

As use herein, “sequence identity” refers to the number of identical amino acids (homology includes conservative amino acid substitutions as well). Sequence identity can be determined by standard alignment algorithm programs, and used with default gap penalties established by each supplier. Substantially homologous nucleic acid molecules would hybridize typically at moderate stringency or at high stringency all along the length of the nucleic acid or along at least about 70%, 80% or 90% of the full length nucleic acid molecule of interest. Also contemplated are nucleic acid molecules that contain degenerate codons in place of codons in the hybridizing nucleic acid molecule. (For proteins, for determination of homology conservative amino acids can be aligned as well as identical amino acids; in this case percentage of identity and percentage homology vary). Whether any two nucleic acid molecules have nucleotide sequences that are at least 80%, 85%, 90%, 95%, 96%, 97%, 98% or 99% “identical” can be determined using known computer algorithms such as the “FAST A “program,” using for example, the default parameters as in Pearson et al. Proc. Natl. Acad. Sci. USA 85: 2444 (1988) (other programs include the GCG program package (Devereux, J., et al., Nucleic Acids Research 12(1): 387 (1984)), BLASTP, BLASTN, FASTA (Atschul, S. F., et al., O. Molec. Biol. 215: 403 (1990); Guide to Huge Computers, Martin J. Bishop, ed., Academic Press, San Diego, 1994, and Carillo et al. SIAM. I. Applied Math 48: 1073 (1988)). For example, the BLAST function of the National Center for Biotechnology Information database can be used to determine identity. Other commercially or publicly available programs include, DNAStar” “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. 0.1. Mol. Biol. 48: 443 (1970), as revised by Smith and Waterman (Adv. Appl. Math. 2: 482 (1981)). Briefly, a GAP program defines similarity as the number of aligned symbols (e.g., nucleotides or amino acids) which are similar, divided by the total number of symbols in the shorter of the two sequences. Default parameters for the GAP program can include: (1) a unary comparison matrix (containing a value of 1 for identities and 0 for non identities) and the weighted comparison matrix of Gribskov et al. Nucl. Acids Res. 14: 6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps. Therefore, as used herein, the term “identity” represents a comparison between a test and a reference polypeptide or polynucleotide.

As used herein, the term “at least 90% identical to” refers to percent identities from 90 to 100% relative to the reference polypeptides. Identity at a level of 90% or more is indicative of the fact that, assuming for exemplification purposes a test and reference polynucleotide length of 100 amino acids are compared, no more than 10% (i.e., 10 out of 100) of amino acids in the test polypeptide differs from that of the reference polypeptides. Similar comparisons can be made between a test and reference polynucleotides. Such differences can be represented as point mutations randomly distributed over the entire length of an amino acid sequence or they can be clustered in one or more locations of varying length up to the maximum allowable, e.g., 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often without relying on software.

As used herein, an “amino acid replacement” refers to the replacement of one amino acid by another amino acid. The replacement can be by a natural amino acid or non-natural amino acids. When one amino acid is replaced by another amino acid in a protein, the total number of amino acids in the protein is unchanged.

As used herein, the phrase “pseudo-wild type,” in the context of single or multiple amino acid replacements, are those amino acids that, while different from the original, such as native, amino acid at a given amino acid position, can replace the native one at that position without introducing any measurable change in a particular protein activity. A population of sets of nucleic acid molecules encoding a collection of mutant molecules is generated and phenotypically characterized such that proteins with sequences of amino acids different from the original amino acid, but that still elicit substantially the same level (i.e., at least 10%, 50%, 70%, 90%, 95%, 100%, depending upon the protein) and type of desired activity as the original protein are selected.

As used herein, “a naked polypeptide chain” refers to a polypeptide that is not post-translationally modified or otherwise chemically modified, and only contains covalently linked amino acids.

As used herein, a “polypeptide complex” includes polypeptides produced by chemical modification or post-translational modification. Such modifications include, but are not limited to, pegylation, albumination, glycosylation, farnysylation, phosphorylation, γ-carboxylation of glutamic acid residues, and/or other polypeptide modifications known in the art.

As used herein, the amino acids, which occur in the various sequences of amino acids provided herein, are identified according to their known, three-letter or one-letter abbreviations.

As used herein, “naturally-occurring” amino acids refer to the 20 L-amino acids that occur in polypeptides. As used herein, the term “non-natural amino acid” refers to an organic compound that has a structure similar to a natural amino acid but has been modified structurally to mimic the structure and reactivity of a natural amino acid. Non-naturally occurring amino acids, thus, include amino acids or analogs of amino acids other than the 20 naturally-occurring amino acids and include, but are not limited to, the D-isostereomers of amino acids.

As used herein, an amino acid is an organic compound containing an amino group and a carboxylic acid group. A polypeptide contains two or more amino acids. For purposes herein, amino acids include the twenty naturally-occurring amino acids non-natural amino acids, and amino acid analogs.

As used herein, an amino acid residue is an amino acid molecule that has lost a hydrogen atom or a hydroxyl moiety by becoming joined to another amino acid molecule. When joined to two other molecules of amino acid(s), the residue has lost both a hydrogen atom and a hydroxyl moiety, thereby having lost a water molecule.

As used herein, “treatment” is an approach for obtaining beneficial or desired clinical results. For purposes of this invention, beneficial or desired clinical results include, but are not limited to, one or more of the following: lessening severity, alleviation, and/or removal of one or more symptoms associated with infection. Treatment also encompasses any pharmaceutical use of the chimeric proteins and compositions provided herein.

As used herein, the “Gla-domain” is an amino acid sequence, usually containing from about 26 to about 45 amino acids, and usually but not always located towards the amino terminal region of a protein, that contains between three and twelve glutamyl residues that are post-translationally modified to γ-carboxyglutamyl residues (Gla). In some cases, the Gla-domain may be defined by exon-intron boundaries of the genomic sequence. The γ-carboxyglutamyl residues in Gla-domains facilitate the calcium-mediated binding of vitamin K-dependent proteins to membrane phospholipids. Prothrombin has a Gla-domain that is encoded within Exon II of the genomic sequence.

### DESCRIPTION

The present invention is based on the discovery that APC inhibits the LPS-mediated release of HMGB1 as well as the HMGB1-mediated pro-inflammatory signaling responses in endothelial cells through down-regulation of the cell surface expression of HMGB1 receptors TLR2, TLR4 and RAGE by EPCR and PAR-1 dependent mechanisms. This finding strongly suggests that the inhibition by APC of this late acting inflammatory mediator may be exerted through its binding of EPCR and contribute to its mortality-reducing, anti-inflammatory protective activity against sepsis.

Moreover, and importantly, it was discovered that the above anti-inflammatory effect of APC is mediated by the Gla-domain of APC through its ability to bind EPCR and the subsequent activation of PAR-1. By contrast, thrombin and meizothrombin can activate PAR-1 at a rate about 1000 times faster than APC, but, because they do not feature the Gla-domain of APC, cannot bind EPCR. This led to the hypothesis that the mutant PCgla/MeizoTh, because of its efficacy in cleaving PAR-1 compounded with its ability to bind EPCR, might have a protective and anti-inflammatory activity similar to that of APC.

Indeed, it was discovered that, unlike meizothrombin, which up-regulates the HMGB1 signaling pathway, the mutant PCgla/MeizoTh inhibits the expression of HMGB1 and its signaling function through the same cell surface receptors as APC, but with about 20 to 50-fold higher efficacy than APC. It was also discovered that, similar to thrombin, but unlike APC, PCgla/MeizoTh in complex with TM cleaves HMGB1 to down-regulate its pro-inflammatory signaling activities by a proteolytic pathway. This is in agreement with previous results showing that the thrombin-cleaved HMGB1 has significantly decreased pro-inflammatory properties. Unlike thrombin, however, PCgla/MeizoTh has minimal procoagulant activity since, unlike thrombin, it cannot effectively cleave fibrinogen.

In view of the above, PCgla/MeizoTh has several advantages as a therapeutic molecule as it exerts the combined effects of inhibiting the expression and signaling effect of HMBG1 and proteolytically cleaving HMBG1 in a reaction that can be markedly accelerated by TM as a cofactor, thereby inhibiting the release of HMGB1 from cells and reducing its amount in plasma. Such advantages, compounded with its high efficacy, render PCgla/MeizoTh an excellent pharmaceutical tool for treating inflammation, especially when it is caused by infection.

Methods of Treatment

In view of the above, there are provided methods for prevention and treatment of inflammation in individuals in need thereof, including immune-compromised patients such as surgical and other hospitalized patients, low birth weight infants, and burn and trauma victims. In particular, the treatment of individuals having symptoms of a systemic septic reaction is contemplated. In some aspects, compositions for preventing and treating sepsis are provided, the compositions comprising a chimeric protein that includes a prothrombin sequence and a protein C (PC) Gla-domain sequence, such as PCgla/MeizoTh. In other aspects, methods of relieving symptoms of and rescuing individuals from episodes of acute septicemia and septic shock utilizing the chimeric protein are provided. The chimeric protein may for instance be administered to treat sepsis due to response to a confirmed infection, such as bacterial infection. Illustrative examples of the chimeric protein and compositions thereof are provided below.

Chimeric Proteins Comprising a Prothrombin Sequence and an PC Gla-Domain Sequence

In representative aspects, the chimeric protein is characterized by having an activity of treating inflammation but also by having a procoagulant activity substantially reduced with respect to the procoagulant activity of naturally occurring, wild-type thrombin, or having substantially no procoagulant activity. Representative chimeric proteins include precursor forms and mature forms; modifications are described with respect to the mature form, but also include modified precursor polypeptides. Corresponding positions on a particular polypeptide may be determined, for example, by alignment of unchanged residues.

In some aspects, the chimeric protein comprises: (a) a prothrombin sequence, and (b) a PC sequence, wherein the chimeric protein can be used to treat inflammation while having substantially no procoagulant activity. In representative examples, the prothrombin sequence comprises one or more fragments of prothrombin and one or more fragments of PC. Fragments from prothrombin may include the prothrombin proteinase domain (PD) and non-catalytic domains Kringle-1 (K1) and Kringle-2 (K2), all may all be part of the prothrombin fragment polypeptide of SEQ ID NO.:3. The PC sequence includes one or more fragments of PC, such as the PC Gla-domain, the PC hydrophobic stack, and, when present in the chimeric protein, the PC pre-pro peptide. The PC fragments may all be part of the PC fragment polypeptide chain of SEQ ID: NO. 4.

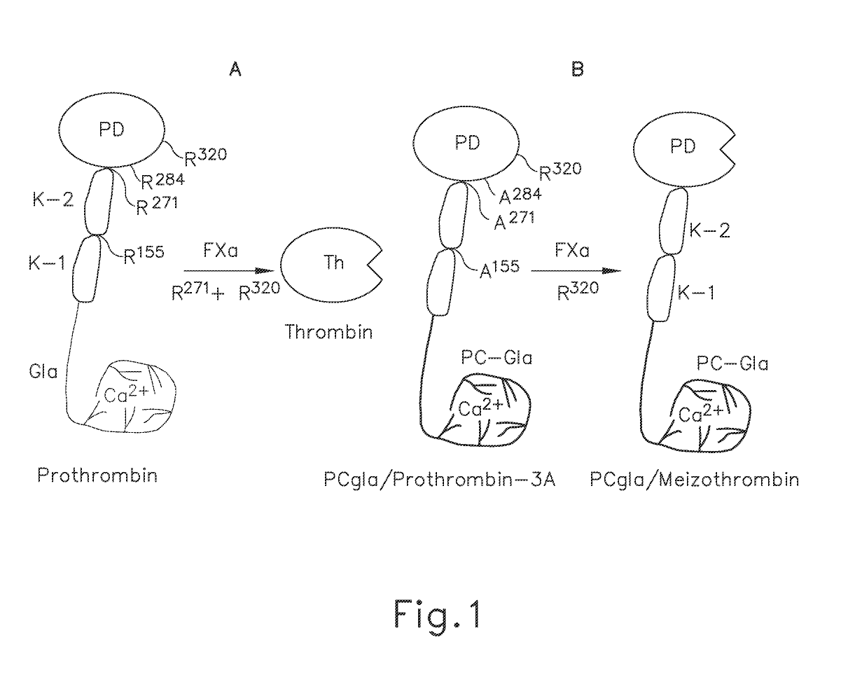

An exemplary chimeric protein is illustrated in FIG. 1B and comprises the polypeptide sequence of SEQ ID NO:5. In other instances, the Gla-domain may be that of PC, and one or both of the prepro leader sequence and hydrophobic stack is from prothrombin.

The procoagulant activity of the chimeric protein may be reduced or substantially eliminated as a result of modifications such as amino acid replacement by means of mutagenesis. This may be achieved by subjecting the prothrombin sequence of the chimeric protein to mutations that render it impervious to cleavage at the locus corresponding to Arg-271 of wild-type prothrombin by Factor Xa (see FIG. 1B). Exemplary among such mutants are chimeric proteins containing an amino acid replacement at said locus of a human prepro prothrombin compared to unmodified human prepro prothrombin, where the human prepro prothrombin comprises a sequence of amino acid residues as set forth in SEQ ID NO:1. In some representative instances, the amino acid residue replacing said Arg is selected from the group consisting of Ala, Val, Leu, Ile, Met, Gln, Glu, Gly, His, Met, Ser, Thr, Trp, and Tyr. An uncharged or hydrophobic residue, such as Ala, Val, Leu, Ile, or Met, is preferred. For example, in polypeptides comprising the sequence of SEQ ID. NO:5, the residue at the locus corresponding to Arg-271 of SEQ ID. NO:1 is replaced with an Ala residue.

Modifications intended to prevent further cleavages of the prothrombin sequence can also be included. In representative instances, the cleavages at the loci corresponding to Arg-155 and Arg-284 of wild-type human prepro prothrombin can be prevented by mutagenesis. Accordingly, the prothrombin sequence of the chimeric protein may contain further amino acid replacements at said loci compared to unmodified human prothrombin. For instance, each of the Arg residues of said loci may be replaced with an amino acid residue selected from the group consisting of Ala, Val, Leu, Ile, Met, Gln, Glu, Gly, H is, Met, Ser, Thr, Trp, and Tyr. An uncharged or hydrophobic residue, such as Ala, Val, Leu, Ile, or Met, is preferred. For example, in polypeptides comprising the sequence of SEQ ID. NO:5, such as “PCgla/Prothrombin-3A,” each of the Arg residues in the loci corresponding to Arg-155, Arg-271 and Arg-284 of SEQ ID NO:1 is replaced with an Ala residue (FIG. 1B).

Accordingly, the chimeric proteins can include a prothrombin sequence which in turn includes an amino acid sequence obtained by deletion, replacement, addition, or insertion of at least one amino acid residue of the proteolytic cleavage sites of unmodified prothrombin. The modified chimeric proteins include precursor forms and mature forms; modifications are described with respect to the mature form, but also include modified precursor polypeptides, such as prepro prothrombins. Corresponding positions on a particular polypeptide may be determined, for example, by alignment of unchanged residues. It is to be understood that there are allelic variants, species variants and isoforms of the polypeptide whose sequences are set forth in SEQ ID NO:1-5, and such polypeptides also can be modified at loci corresponding to those of the polypeptides exemplified herein.

In some examples, the chimeric protein includes a polypeptide comprising an amino acid sequence of Formula (I):

(1-88)-(89-197)-X₁-(199-313)-X₂-(315-326)-X₃-(328-622) Formula (1)

wherein

each of X₁, X₂ and X₃ is an amino acid residue independently selected from the group consisting of Ala, Val, Leu, Ile, Met, Gln, Glu, Gly, His, Met, Ser, Thr, Trp, and Tyr, (1-88) is a peptide chain including the first 88 amino acid residues of the human prepro PC polypeptide counted from the N-terminal end, such as the sequence of SEQ ID NO: 4 or an analog thereof or derivative thereof, and the peptide chain stretching from residue 89 to 622 includes amino acids corresponding to amino acids 89 to 622 of SEQ ID NO:1, or an analog thereof or derivative thereof. The Arg residues of positions 198, 314, and 327 of said chain have been mutated to X₁, X₂, and X₃, respectively.

In some cases, the chimeric protein is a naked polypeptide chain. In other cases, the polypeptide is a polypeptide complex further comprising post-translational modifications, such as γ-carboxylated glutamic acid residues. The sequences SEQ ID NO: 1-5 and of Formula (1) are therefore to be understood as including such γ-carboxylations at the appropriate loci of their respective Gla-domains. The polypeptide may also include other modifications known in the art. Hence, the chimeric polypeptide may also be pegylated, albuminated, glycosylated, lipidated, form disulfide bridges, or has undergone other modifications, such as the cleavage leading from prothrombin to meizothrombin. Also contemplated are modified chimeric proteins further having one or more pseudo-wild-type mutations. Representative pseudo-wild-type mutations include deletion, replacement, addition, insertion or a combination thereof of the amino acid residue(s) of an unmodified chimeric protein.

Nucleic Acid Molecules and Vectors

In another aspect, there are provided nucleic acid molecules comprising polynucleotide sequences which code for a chimeric polypeptide as described herein. Also provided are vectors comprising such polynucleotide sequences and host cells containing such nucleic acid molecules or vectors. The chimeric polypeptide may be produced by expressing a polynucleotide sequence encoding the chimeric polypeptide in a suitable host cell by standard techniques. The chimeric polypeptide is either expressed directly or as a precursor molecule which has an N-terminal or C-terminal extension, such as a His-tag. Polynucleotide sequences coding for the chimeric polypeptide may be prepared synthetically by established standard methods, e.g. the phosphoramidite method with an automatic DNA synthesizer and/or by polymerase chain reaction (PCR).

In a further aspect, there is provided a vector which is capable of replicating in a selected microorganism or host cell and which carries a polynucleotide sequence encoding a chimeric protein as described herein. The recombinant vector may be an autonomously replicating vector, i.e., a vector which exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome. In one representative instance, the recombinant expression vector is capable of replicating in yeast. Examples of sequences which enable the vector to replicate in yeast are the yeast plasmid 2 um replication genes REP 1-3 and origin of replication.

The vectors may contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance. Selectable markers for use in a filamentous fungal host cell include amdS (acetamidase), argB (ornithine carbamoyltransferase), pyrG (orotidine-5′-phosphate decarboxylase) and trpC (anthranilate synthase). Suitable markers for yeast host cells are ADE2, HIS3, LEU2, LYS2, MET3, TRP1, and URA3. A well suited selectable marker for yeast is the Schizosaccharomyces pombe TPI gene (Russell (1985) Gene 40:125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intra-cellular polypeptides either homologous or heterologous to the host cell. Examples of suitable promoters for directing the transcription in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), and Bacillus licheniformis penicillinase gene (penP). Examples of suitable promoters for directing the transcription in a filamentous fungal host cell are promoters obtained from the genes for Aspergillus oryzae TAKA amylase, Rhizomucor miehei aspartic proteinase, Aspergillus niger neutral alpha-amylase, and Aspergillus niger acid stable alpha-amylase. In a yeast host, useful promoters are the Saccharomyces cerevisiae Mai, TPI, ADH or PGK promoters. The polynucleotide construct will also typically be operably connected to a suitable terminator. In yeast a suitable terminator is the TPI terminator (Alber et al. (1982) J. Mol. Appl. Genet. 1:419-434).

The procedures used to ligate the polynucleotide sequence, the promoter and the terminator, respectively, and to insert them into a suitable vector containing the information necessary for replication in the selected host, are well known to those skilled in the art. It will be understood that the vector may be constructed either by first preparing a DNA construct containing the entire DNA sequence encoding a chimeric polypeptide as described herein, and subsequently inserting this fragment into a suitable expression vector, or by sequentially inserting DNA fragments containing genetic information for individual elements of the chimeric polypeptide followed by ligation.

Also provided are recombinant host cells, comprising a polynucleotide sequence encoding a chimeric polypeptide as described herein. A vector comprising such polynucleotide sequence is introduced into the host cell so that the vector is maintained as a chromosomal integrant or as a self-replicating extra-chromosomal vector as described earlier. The host cell may be a unicellular microorganism, e.g., a prokaryote, or a non-unicellular microorganism, e.g., a eukaryote. Example host cells include bacterial cells, insect cells, mammalian cells, plant cells, and yeast cells.

Pharmaceutical Compositions

Pharmaceutical compositions comprising a chimeric polypeptide as described herein can be used in the treatment of conditions which are sensitive to antiinflammatories.

The optimal dose level for any patient will depend on a variety of factors including the efficacy of the specific chimeric polypeptide as described herein employed, the age, body weight, physical activity, and diet of the patient, on a possible combination with other drugs, and on the severity of the condition to be treated. It is recommended that the daily dosage of a composition be determined for each individual patient by those skilled in the art in a clinical setting.

Pharmaceutical compositions of a chimeric polypeptide as described herein may contain adjuvants and additives typical of pharmaceutical formulations and are usually formulated as an aqueous solution. The aqueous medium may be made isotonic, for example, with sodium chloride, sodium acetate or glycerol. Furthermore, the aqueous medium may contain pH-adjusting additives such as buffers, and preservatives. Consequently, there is also provided a pharmaceutical composition comprising a chimeric polypeptide as described herein and optionally one or more agents suitable for stabilization, preservation or isotonicity, for example, transition metal ions, phenol, cresol, a parabene, sodium chloride, glycerol or mannitol.

The pH-adjusting agent used in the pharmaceutical composition may be a buffer selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof.

The pharmaceutically acceptable preservative may be selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In some cases, the preservative is present in a concentration from 0.1 mg/ml to 20 mg/ml. In some instances, the preservative is present in a concentration from 0.1 mg/ml to 5 mg/ml. In other instances, the preservative is present in a concentration from 5 mg/ml to 10 mg/ml. In further instances, the preservative is present in a concentration from 10 mg/ml to 20 mg/ml. The use of a preservative in pharmaceutical compositions is well-known to the skilled person. For convenience, reference is made to Remington: The Science and Practice of Pharmacy, 19th edition, 1995.

The isotonicity agent may be selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles. In some aspects, more than one chimeric protein as described herein may be administered, for example in compositions that may contain at least one, at least two, at least three, at least four, at least five different modified chimeric polypeptides. A mixture of modified chimeric polypeptides may be particularly useful in treating a broader range of population of individuals.

A polynucleotide encoding a chimeric polypeptide as described herein may also be used for delivery and expression of any of said polypeptides in a desired cell. It is apparent that an expression vector can be used to direct expression of a chimeric polypeptide according to methods known in the art. The expression vector can be administered by any means known in the art, such as intraperitoneally, intravenously, intramuscularly, subcutaneously, intrathecally, intraventricularly, orally, enterally, parenterally, intranasally, dermally, sublingually, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a nucleic acid molecule encoding a chimeric polypeptide as described herein can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. ScL (USA) (1990) 87:3655; Wu et al., J. Biol. Chem. (1991) 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol.

The therapeutic polynucleotides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740; 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu5 J. Biol. Chem. (1989) 264:16985); eukaryotic cell-delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent NO. 0 524 968. Additional approaches are described in Philip, Mol. Cell. Biol. (1994) 14:2411 and in Woffendin, Proc. Natl. Acad. Sci. (1994) 91:1581.

A chimeric polypeptide is included as active compound in a pharmaceutically acceptable carrier in an amount sufficient to exert a therapeutically useful effect. The therapeutically effective concentration can be determined empirically by testing the compounds in known in vitro and in vivo systems. The active compounds can be administered by any appropriate route, for example, orally, nasally, pulmonarily, parenterally, intravenously, intradermally, subcutaneously, or topically, in liquid, semi-liquid or solid form and are formulated in a manner suitable for each route of administration.

The chimeric polypeptide and physiologically acceptable salts and solvates thereof can be formulated for administration by inhalation (either through the mouth or the nose), oral, pulmonary, transdermal, parenteral or rectal administration. For administration by inhalation, the chimeric protein can be delivered in the form of an aerosol spray from pressurized packs or a nebulizer with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of e.g., gelatin for use in an inhaler or insufflator, can be formulated containing a powder mix of a therapeutic compound and a suitable powder base such as lactose or starch.

For pulmonary administration to the lungs, the chimeric protein can be delivered in the form of an aerosol spray from a nebulizer, turbonebulizer, or microprocessor-controlled metered dose oral inhaler with the use of a suitable propellant. Usually, the particle size of the aerosol spray is small, such as in the range of 0.5 to 5 microns. In the case of a pharmaceutical composition formulated for pulmonary administration, detergent surfactants are not typically used.

The chimeric polypeptide can be formulated as a depot preparation. Such long-acting formulations can be administered by implantation (for example, subcutaneously or intramuscularly) or by intramuscular injection. Thus, for example, the therapeutic compounds can be formulated with suitable polymeric or hydrophobic materials (for example, as an emulsion in an acceptable oil), ion exchange resins, or as sparingly soluble derivatives, for example, as a sparingly soluble salt.

The chimeric polypeptide can be formulated for parenteral administration by injection (e.g., by bolus injection or continuous infusion). Formulations for injection can be presented in unit dosage form (e.g., in ampoules or in multi-dose containers) with an added preservative. The compositions can take such forms as suspensions, solutions or emulsions in oily or aqueous vehicles and can contain formulatory agents such as suspending, stabilizing and/or dispersing agents. Alternatively, the active ingredient can be in powder-lyophilized form for constitution with a suitable vehicle, e.g., sterile pyrogen free water, before use.

The chimeric polypeptide can also be formulated for local or topical application, such as for topical application to the skin (transdermal) and mucous membranes, such as in the eye, in the form of gels, creams, and lotions and for application to the eye or for intracistemal or intraspinal application. Such solutions, particularly those intended for ophthalmic use, can be formulated as 0.01%-10% isotonic solutions and pH about 5-7 with appropriate salts. The compounds can be formulated as aerosols for topical application, such as by inhalation.

The concentration of active compound in a pharmaceutical composition depends on absorption, inactivation and excretion rates of the active compound, the dosage schedule, and amount administered as well as other factors known to those of skill in the art. The pharmaceutical compositions, if desired, can be presented in a package, in a kit or dispenser device, that can contain one or more unit dosage forms containing the active ingredient. The package, for example, contains metal or plastic foil, such as a blister pack. The pack or dispenser device can be accompanied by instructions for administration. The pharmaceutical compositions containing the active agents can be packaged as articles of manufacture containing packaging material, an agent provided herein, and a label that indicates the disorder for which the agent is provided.

For oral administration, the pharmaceutical compositions can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or Wetting agents (e.g., sodium lauryl sulphate). The tablets can be coated by methods well known in the art. Liquid preparations for oral administration can take the form of, for example, solutions, syrups or suspensions, or they can be presented as a dry product for constitution with Water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol or fractionated vegetable oils); and preservatives (e.g., methyl or propyl p hydroxybenzoates or sorbic acid). The preparations also can contain buffer salts, flavoring, coloring and/or sweetening agents as appropriate.

Also provided are pharmaceutical compositions of nucleic acid molecules encoding the chimeric polypeptide and expression vectors encoding them that are suitable for gene therapy. Rather than deliver the protein, nucleic acid molecules can be administered in vivo (e.g., systemically or by other routes), or ex vivo, such as by removal of cells, including lymphocytes, introduction of the nucleic acid molecule therein, and reintroduction into the host or a compatible recipient. Accordingly, a chimeric polypeptide can be delivered to cells and tissues by expression of nucleic acid molecules. The chimeric polypeptide can be administered as nucleic acid molecules encoding the chimeric polypeptide, including ex vivo techniques and direct in vivo expression.

Nucleic acid molecules can be delivered to cells and tissues by any method known to those of skill in the art. The isolated nucleic acid molecules can be incorporated into vectors for further manipulation. As used herein, vector (or plasmid) refers to discrete elements that are used to introduce heterologous DNA into cells for either expression or replication thereof. Methods for administering chimeric polypeptide by expression of encoding nucleic acid molecules include administration of recombinant vectors. The vector can be designed to remain episomal, such as by inclusion of an origin of replication or can be designed to integrate into a chromosome in the cell. Chimeric polypeptides also can be used in ex vivo gene expression therapy using non-viral vectors. For example, cells can be engineered to express a chimeric polypeptide, such as by integrating a chimeric protein-encoding nucleic acid molecule into a genomic location, either operatively linked to regulatory sequences or such that it is placed operatively linked to regulatory sequences in a genomic location. Such cells then can be administered locally or systemically to a subject, such as a patient in need of treatment.

Viral vectors, include, for example adenoviruses, herpes viruses, retroviruses and others designed for gene therapy can be employed. The vectors can remain episomal or can integrate into chromosomes of the treated subject. A chimeric polypeptide can be expressed by a virus, which is administered to a subject in need of treatment. Virus vectors suitable for gene therapy include adenovirus, adeno-associated virus, retroviruses, lentiviruses and others noted above. For example, adenovirus expression technology is well-known in the art and adenovirus production and administration methods also are well known. Adenovirus serotypes are available, for example, from the American Type Culture Collection (ATCC, Rockville, Md.). Adenovirus can be used ex vivo. For example, cells are isolated from a patient in need of treatment, and transduced with a chimeric polypeptide-expressing adenovirus vector. After a suitable culturing period, the transduced cells are administered to a subject locally and/or systemically. Alternatively, chimeric polypeptide-expressing adenovirus particles are isolated and formulated in a pharmaceutically-acceptable carrier for delivery of a therapeutically effective amount to prevent, treat or ameliorate a disease or condition of a subject. In some situations it is desirable to provide a nucleic acid molecule source with an agent that targets cells, such as an antibody specific for a cell surface membrane protein or a target cell, or a ligand for a receptor on a target cell.

The nucleic acid molecules can be introduced into artificial chromosomes and other non-viral vectors. Artificial chromosomes, such as ACES (see, Lindenbaum et al. Nucleic Acids Res. 32(21): e172 (2004)) can be engineered to encode and express the isoform. Briefly, mammalian artificial chromosomes (MACs) provide a means to introduce large payloads of genetic information into the cell in an autonomously replicating, non-integrating format. Unique among MACs, the mammalian satellite DNA-based Artificial Chromosome Expression (ACE) can be reproducibly generated de novo in cell lines of different species and readily purified from the host cells' chromosomes. Purified mammalian ACEs can then be re-introduced into a variety of recipient cell lines where they have been stably maintained for extended periods in the absence of selective pressure using an ACE System. Using this approach, specific loading of one or two gene targets has been achieved in LMTK(−) and CHO cells.

In yet another method is a two-step gene replacement technique in yeast, starting with a complete adenovirus genome (Ad2; Ketner et al. Proc. Natl. Acad. Sci. USA 91: 6186-6190 61 (1994)) cloned in a Yeast Artificial Chromosome (YAC) and a plasmid containing adenovirus sequences to target a specific region in the YAC clone, an expression cassette for the gene of interest and a positive and negative selectable marker.

The nucleic acids encoding the chimeric polypeptides can be encapsulated in a vehicle, such as a liposome, or introduced into a cell, such as a bacterial cell, particularly an attenuated bacterium or introduced into a viral vector. For example, when liposomes are employed, proteins that bind to a cell surface membrane protein associated with endocytosis can be used for targeting and/or to facilitate uptake, e.g., capsid proteins or fragments thereof tropic for a particular cell type, antibodies for proteins which undergo internalization in cycling, and proteins that target intracellular localization and enhance intracellular half-life.

For ex vivo and in vivo methods, nucleic acid molecules encoding the chimeric polypeptides are introduced into cells that are from a suitable donor or the subject to be treated. Cells into which a nucleic acid molecule can be introduced for purposes of therapy include, for example, any desired, available cell type appropriate for the disease or condition to be treated, including but not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof.

For ex vivo treatment, cells from a donor compatible with the subject to be treated or the subject to be treated cells are removed, the nucleic acid is introduced into these isolated cells and the modified cells are administered to the subject. Treatment includes direct administration, such as, for example, encapsulated within porous membranes, which are implanted into the patient. Techniques suitable for the transfer of nucleic acid into mammalian cells in vitro include the use of liposomes and cationic lipids (e.g., DOTMA, DOPE and DCChoI) electroporation, microinjection, cell fusion, DEAE-dextran, and calcium phosphate precipitation methods. Methods of DNA delivery can be used to express chimeric polypeptides in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

In vivo expression of a chimeric polypeptide can be linked to expression of additional molecules. For example, expression of a chimeric polypeptide can be linked with expression of a cytotoxic product such as in an engineered virus or expressed in a cytotoxic virus. Such viruses can be targeted to a particular cell type that is a target for a therapeutic effect. The expressed chimeric polypeptide can be used to enhance the cytotoxicity of the virus. In vivo expression of a chimeric polypeptide can include operatively linking a chimeric polypeptide encoding nucleic acid molecule to specific regulatory sequences such as a cell-specific or tissue-specific promoter. Chimeric polypeptides also can be expressed from vectors that specifically infect and/or replicate in target cell types and/or tissues. Inducible promoters can be use to selectively regulate chimeric polypeptide expression.

Nucleic acid molecules in the form of naked nucleic acids or in vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell. Administration also can be direct, such as by administration of a vector or cells that typically targets a cell or tissue. For example, tumor cells and proliferating can be targeted cells for in vivo expression of chimeric polypeptides. Cells used for in vivo expression of a chimeric polypeptide also include cells autologous to the patient. These cells can be removed from a patient, nucleic acids for expression of a chimeric polypeptide introduced, and then administered to a patient such as by injection or engraftment.

Polynucleotides and expression vectors provided herein can be made by any suitable method. Further provided are nucleic acid vectors containing nucleic acid molecules as described above, including a nucleic acid molecule containing a sequence of nucleotides that encodes the polypeptide as set forth in any of SEQ ID NO.: 3 or a functional fragment thereof. Further provided are nucleic acid vectors containing nucleic acid molecules as described above and cells containing these vectors.

Therapeutic Uses

The chimeric polypeptides and nucleic acid molecules provided herein can be used for treatment of conditions where HMGB1-based signaling is believed to be involved. This section provides exemplary uses of the chimeric polypeptides and administration methods. Such methods include, but are not limited to, methods of treatment of physiological and medical conditions described and listed below. In particular, the chimeric polypeptides are intended for use in therapeutic methods for the treatment of sepsis. The chimeric polypeptides and nucleic acid molecules encoding the chimeric polypeptides also can be administered in combination with other therapies including other biologics and small molecule compounds.

Treatment of diseases and conditions with the chimeric polypeptides can be effected by any suitable route of administration using suitable formulations as described herein, including but not limited to, subcutaneous injection, oral, nasal, pulmonary and transderrnal administration. If necessary, a particular dosage and duration and treatment protocol can be empirically determined or extrapolated. For example, exemplary doses of chimeric polypeptides can be used as a starting point to determine appropriate dosages. Particular dosages and regimens can be empirically determined.

Dosage levels would be apparent to one of skill in the art and would be determined based on a variety of factors, such as body weight of the individual, general health, age, the activity of the specific compound employed, sex, diet, time of administration, rate of excretion, drug combination, the severity and course of the disease or condition, and the subject's disposition to the disease/condition and the judgment of the treating physician. The amount of active ingredient that can be combined with the carrier materials to produce a single dosage form may vary depending upon the subject treated and the particular mode of administration. Upon improvement of a subject's condition, a maintenance dose of a compound or composition provided herein can be administered, if necessary; and the dosage, the dosage form, or frequency of administration, or a combination thereof, can be varied. In some cases, the subject can require intermittent treatment on a long-term basis upon any recurrence of disease symptoms.

Administration of a chimeric polypeptide can be continuous or intermittent, depending, for example, upon the recipient's physiological condition, whether the purpose of the administration is therapeutic or prophylactic, and other factors known to skilled practitioners. The administration of may be essentially continuous over a preselected period of time or may be in a series of spaced dosages, e.g., either before, during, or after the insurgence of sepsis. Also provided herein is the use of any of the chimeric polypeptides provided herein for the manufacture of a medicament for treating of a subject having inflammation due to infection.

### Experimental

### Materials and Methods

Reagents

Bacterial lipopolysaccharide (LPS), 2-mercaptoethanol and antibiotics (penicillin G and streptomycin) were purchased from Sigma (St. Louis, Mo.). Human recombinant HMGB1 was purchased from Abnova (Taipei City, Taiwan). Fetal bovine serum (FBS) and Vybrant DiD were purchased from Invitrogen (Carlsbad, Calif.). The cleavage-blocking monoclonal anti-PAR-1 antibody (H-111) was purchased from Santa Cruz Biotechnology Inc. (Santa Cruz, Calif.). The function-blocking anti-EPCR antibody was purchased from Cell Sciences (Canton, Mass.). All recombinant proteins including APC and the thrombin activation intermediate meizothrombin (MeizoTh) and PCgla/MeizoTh were prepared as described (see FIG. 1). [26]

Cell Culture

Primary HUVECs were obtained from Cambrex Bio Science Inc. (Charles City, Iowa) and maintained as described. Human monocytic leukemia cell line, THP-1, was maintained at a density of 2×10⁵ to 1×10⁶ cells/mL in RPMI 1640 with L-glutamine and 10% heat-inactivated FBS supplemented with 2-mercaptoethanol (55 μM) and antibiotics (penicillin G and streptomycin, as described. [27]

ELISAs for HMGB1, NF-κB and TNF-α

Commercially available ELISA kits were used to measure the concentrations of HMGB1 (Shino-Test Corporation, Tokyo, Japan), NF-κB (Cell Signaling Technology, Inc, Danvers, Mass.) and TNF-α (R&D Systems, Minneapolis, Minn.) in cell culture supernatants according to the manufacturers' protocols.

Cell Adhesion Assay

THP-1 cell adherence to endothelial cells was evaluated by fluorescent labeling of THP-1 cells as described. [28] Briefly, THP-1 cells were labeled with the Vybrant DiD dye followed by their addition to the washed and stimulated HUVECs. Cells were allowed to adhere and the non-adherent THP-1 cells were washed off and the fluorescence of the adherent cells was measured. The percentage of adherent THP-1 cells was calculated by the formula: % adherence=(adherent signal/total signal)×100 as described. [28] The data are expressed as means±S.D. from at least three independent experiments.

Migration Assay

The migration assay was performed in trans-well plates of 6.5 mm diameter, with 8 μm pore size filters as described. [27,29] HUVECs (6×10⁴) were cultured for three days to obtain confluent endothelial cell monolayers. The monolayers were treated for 3 hours with indicated proteases followed by HMGB1 (1 μg/mL for 16 h) and washed three times with PBS, and THP-1 cells were immediately added to the upper compartment. After trans-well plates were incubated for 2 hours, cells in the upper chamber of the filter were aspirated and the non-migrating cells on top of the filter were removed with a cotton swab. THP-1 cells on the lower side of the filter were fixed with 7-8% glutaraldehyde and stained with 0.25% crystal violet in 20% methanol (w/v). Each experiment was repeated in duplicate wells and within each well counting was done in nine randomly selected microscopic high power fields.

Analysis of Expression of Cell Surface Receptors

The expression of vascular cell adhesion molecule-1 (VCAM-1), intercellular adhesion molecule-1 (ICAM-1) and E-selectin on HUVECs was determined by a whole-cell ELISA as described. [29] Briefly, cell monolayers, which were treated for 3 hours with indicated proteases, were incubated with HMGB1 (1 μg/mL for 16 hours) and then fixed in 1% paraformaldehyde. After washing three times, mouse anti-human monoclonal antibodies to VCAM-1, ICAM-1, and E-selectin (Temecula, Calif., USA) were added. After 1 hours (37° C., 5% CO₂), the cells were washed and peroxidase-conjugated anti-mouse IgG (Sigma, St. Louis, Mo.) was added for 1 hours. The cells were washed again and developed using o-phenylenediamine substrate (Sigma, St. Louis, Mo.). All measurements were performed in triplicate wells. The same experimental procedures were used to monitor the cell surface expression of TLR2, TLR4 and RAGE receptors using specific antibodies (A-9, H-80 and A-9, respectively) obtained from Santa Cruz Biotechnology Inc. (Santa Cruz, Calif.).

RNA Interference

The expression of inflammatory mediators (NF-κB and TNF-α) by endothelial cells in response to HMGB1 (1 μg/mL for 16 h) was evaluated following the knockdown of TLR2, TLR4 and RAGE expression by pools of target-specific 20-25 nucleotide siRNAs obtained from Santa Cruz Biotechnology Inc. (Santa Cruz, Calif.) according to the manufacturer's instruction and as described. [27] A non-targeting 20-25 nucleotide siRNA obtained from the same company was used as a negative control.

HMGB1 Degradation Assay

The degradation of HMGB1 by PCgla/MeizoTh was monitored by SDS-PAGE as described. [25] Briefly, HMGB1 (400 nmol/L) was incubated with 2 nM PCgla/MeizoTh with or without 400 nmol/L recombinant TM for 15 to 120 minutes at 37° C. in 50 mmol/L Tris-HCl (pH 8.0), 2 mmol/L CaCl₂, and 0.1 mol/L NaCl in a total volume of 50 μL. [25] These samples were then run on SDS PAGE (10%, reducing) followed by immunoblot analysis using a rabbit polyclonal antibody against HMGB1 (Abcam, UK).

Statistical Analysis

Data are expressed as means±standard deviations from at least three independent experiments. Statistical significance between 2 groups was determined by Student's t-test. The significance level was set at p<0.05.

Results

Effect of APC on the LPS-Induced HMGB1 Release

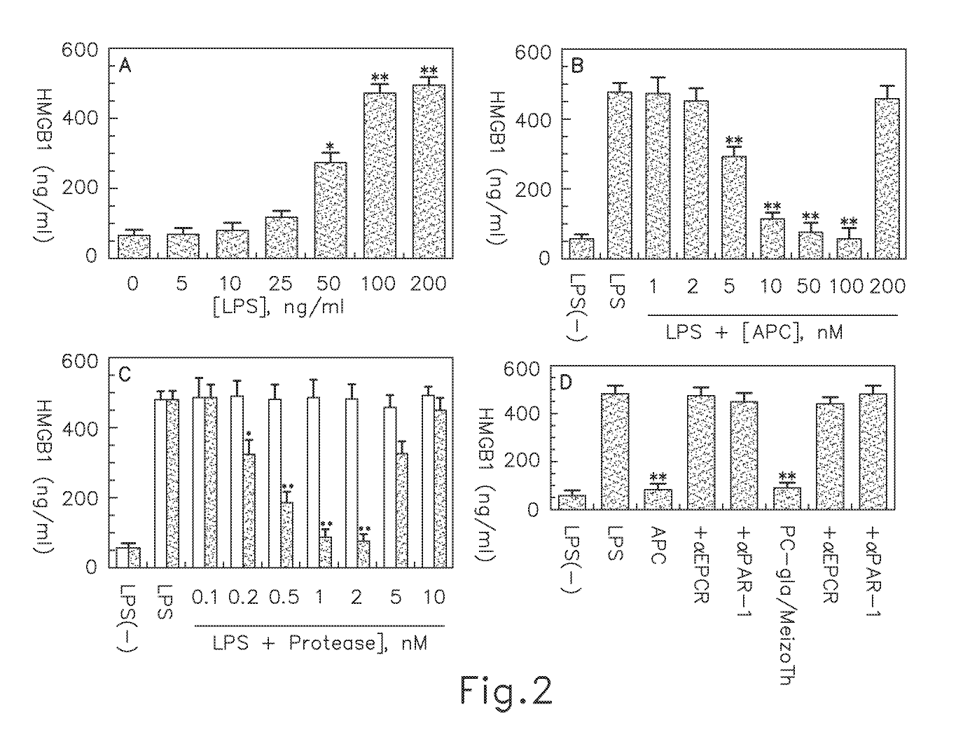

Previous studies have demonstrated that HMGB1 can be released from human endothelial cells in response to both endotoxin and TNF-α. [9-11] Following its release to intravascular spaces, HMGB1 is known to interact with specific cell surface receptors to amplify inflammatory responses by inducing the expression of pro-inflammatory cytokines. [8-11] In agreement with previous results, the experiments reported herein show that LPS stimulated HMGB1 release by HUVECs by a concentration dependent manner (FIG. 2A). The LPS-mediated HMGB1 release occurred with late kinetics of about 8 hours after stimulation by LPS and reached its peak level at about 16 hours after LPS stimulation (data not shown).

To investigate the effect of APC on the LPS-mediated HMGB1 release, endothelial cells were pretreated with increasing concentrations of APC for 3 hours before stimulation of cells with 100 ng/mL LPS for 16 hours. The results presented in FIG. 2B indicated that APC inhibits the HMGB1 release by endothelial cells with an inhibitory effect that could be observed at a concentration of above 10 nM APC. An APC concentration of 100 nM was required to obtain an optimal effect (FIG. 2B) In a previous study, the Gla-domain of the thrombin activation intermediate product, meizothrombin, was replaced with the corresponding domain of APC and it was shown that the resulting mutant protease (PCgla/MeizoTh) elicits a barrier protective effect in endothelial cells in response to pro-inflammatory mediators including LPS and TNF-α. [20,26] The results presented in FIG. 2C demonstrate that PCgla/MeizoTh was also a potent inhibitor of HMGB1 release by endothelial cells with its optimal effect occurring at a concentration of less than 1 nM.

By contrast, wild-type MeizoTh exhibited no protective activity in this assay (FIG. 2C, white bars), suggesting that the interaction of the Gla-domain of APC with EPCR is a prerequisite for the protective activity of the mutant protease. The inhibitory effects of both proteases toward HMGB1 required the EPCR-dependent activation of PAR-1 as evidenced by the function-blocking antibodies to both receptors abrogating the protective effects (FIG. 2D).

Effects of APC and PCgla/MeizoTh on the HMGB1-Mediated Cam Expression, THP-1 Adhesion and Migration

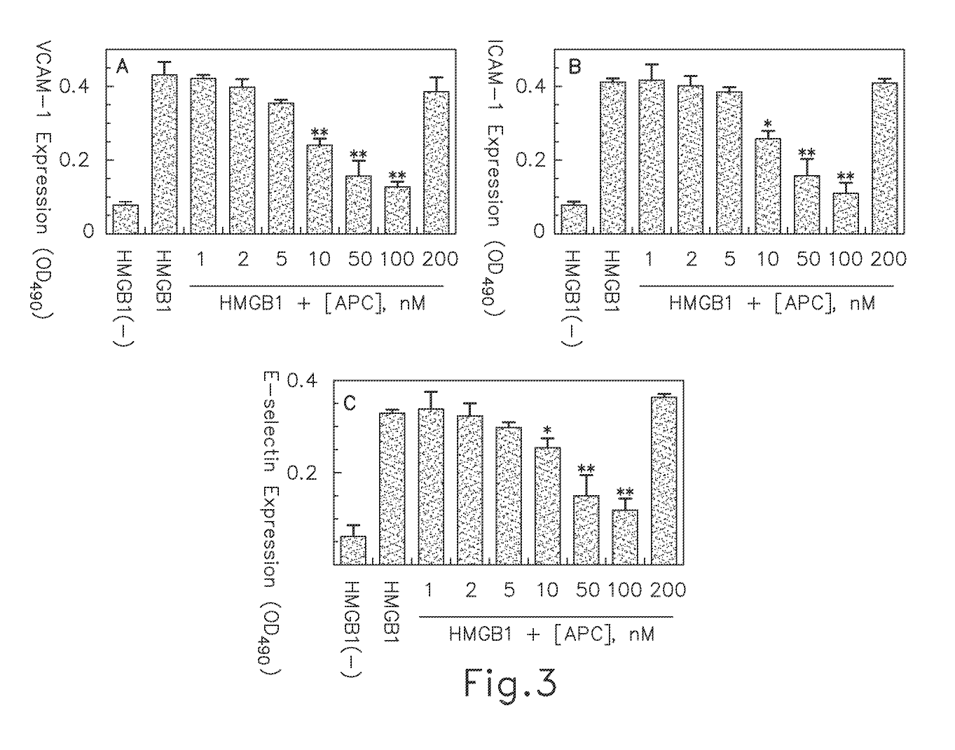

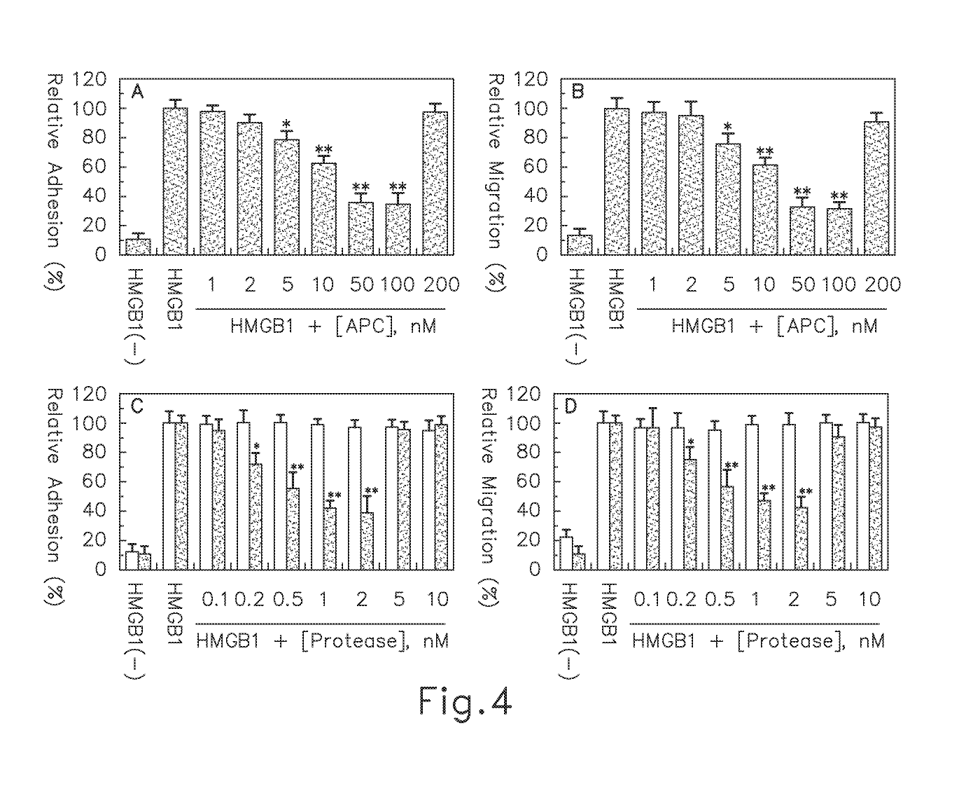

Previous results have indicated that HMGB1 mediates pro-inflammatory responses by increasing the expression of cell adhesion molecules ICAM-1, VCAM-1 and E-selectin on the surface of endothelial cells, thereby promoting the adhesion and migration of leukocytes across the endothelium. [9-11] As presented in FIG. 3, HMGB1 up-regulated the cell surface expression of all three adhesion molecules (CAM) and APC inhibited this effect of HMGB1 by a concentration dependent manner. The inhibitory effect of APC toward the expression of CAMs was mediated through APC down-regulating the HMGB1 signaling pathway. The elevated expression of CAMs correlated well with an enhanced binding of THP-1 cells to the HMGB1-activated endothelial cells and their subsequent migration across the monolayer (FIGS. 4A and B). APC down-regulated the adherence of THP-1 cells and their migration across activated endothelial monolayer by a concentration dependent manner (FIGS. 4A and B).

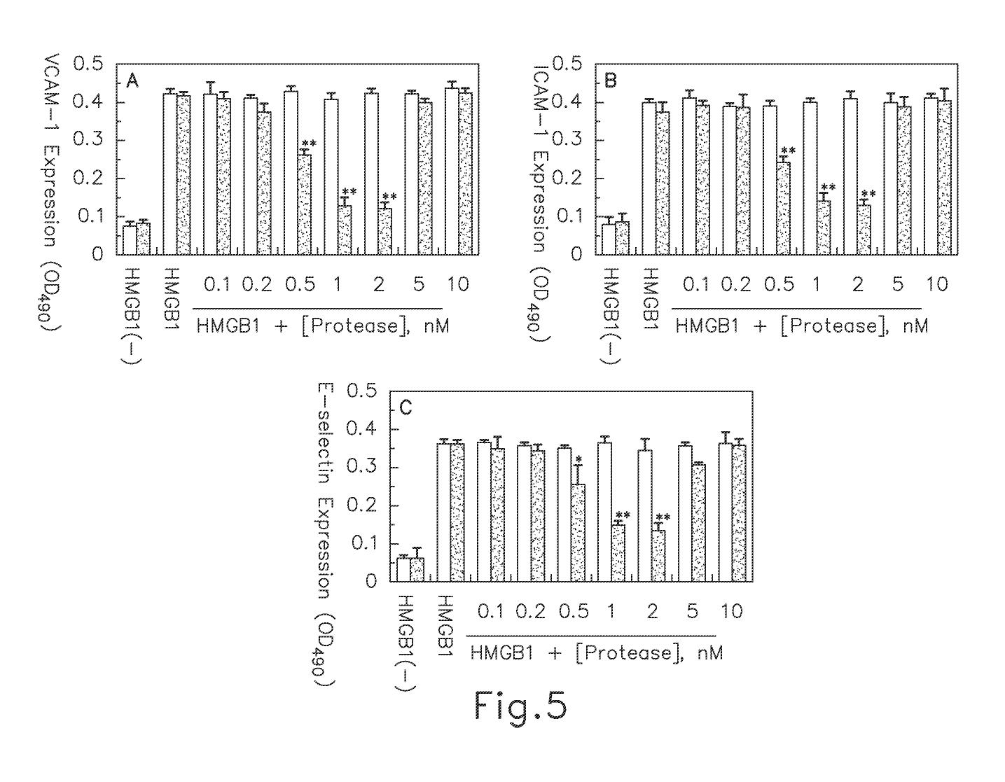

These results suggest that APC not only inhibits the endotoxin-mediated release of HMGB1 by endothelial cells but also down-regulates the pro-inflammatory signaling effect of the released HMGB1, thereby inhibiting the amplification of the pro-inflammatory pathways by the nuclear cytokine. Consistent with the data presented above, PCgla/MeizoTh, but not wild-type MeizoTh, inhibited the cell surface expression of all three CAMs (FIG. 5) and it also inhibited the adherence and migration of THP-1 cells across HMGB1-activated endothelial cell monolayer with about 20 to 50-fold higher efficacy (FIGS. 4C and D). The antiinflammatory effects of both APC and PCgla/MeizoTh required the EPCR-dependent cleavage of PAR-1 since the function-blocking antibodies to both receptors abrogated these effects (data not shown).

Effect of APC and PCgla-MeizoTh on the HMGB1-Mediated NF-κB Activation and TNF-α Expression

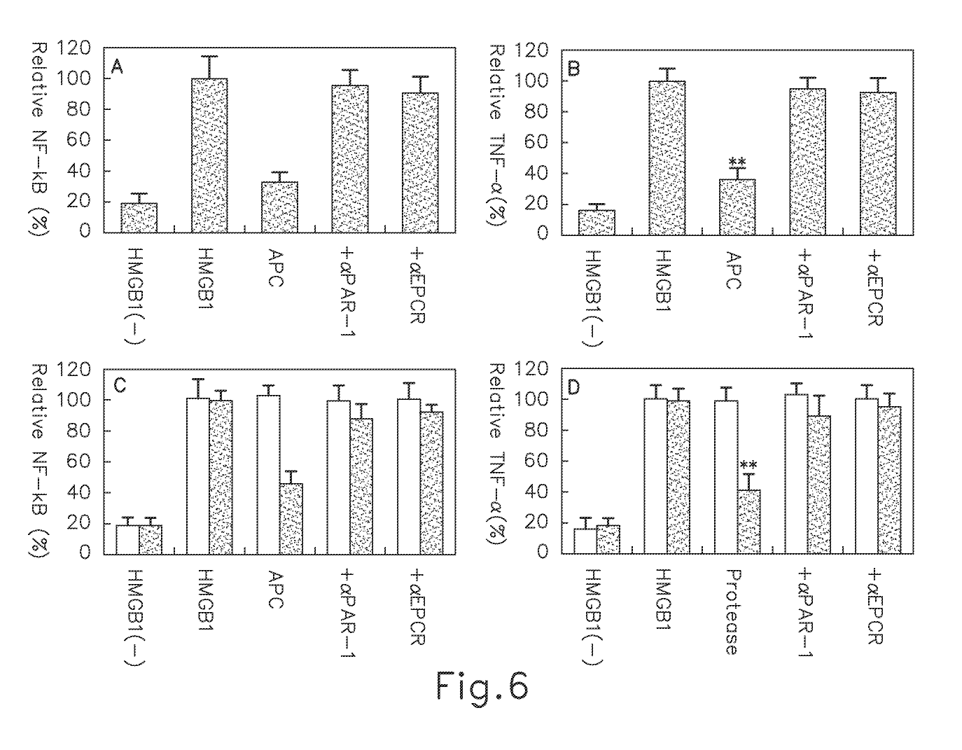

It is known that HMGB1 up-regulates inflammatory pathways by activating NF-κB and promoting the expression of TNF-α by endothelial cells and monocytes. [4, 8, 9] As presented in FIG. 6, HMGB1 activated NF-κB and stimulated the expression of TNF-α by endothelial cells by EPCR and PAR-1 dependent mechanisms. The PCgla/MeizoTh mutant elicited a similar protective effect, but required a significantly lower concentration of the protease to inhibit the HMGB1-mediated activation of NF-κB as well as the expression of TNF-α by endothelial cells (FIGS. 6C and D).

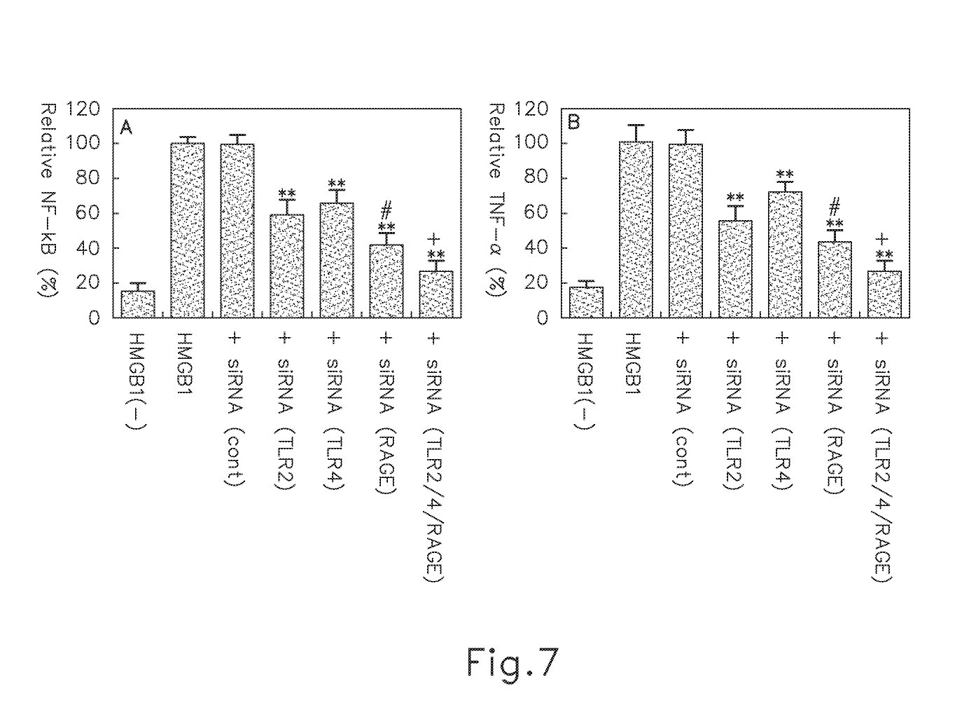

Further studies revealed that the pro-inflammatory activity of HMGB1 is mediated through its interaction and subsequent signaling through at least three cell surface receptors TLR2, TLR4 and RAGE since specific siRNAs for each receptor significantly inhibited both NF-κB activation and TNF-α expression by endothelial cells in response to HMGB1 (FIGS. 7A and B). All three receptors appeared to be involved in HMGB1 signaling in endothelial cells since transfecting cells with the combination of all three siRNAs exhibited an additive effect in inhibiting the inflammatory mediators (FIGS. 7A and B).

APC and PCgla/MeizoTh Down-Regulate the Expression of HMGB1 Receptors

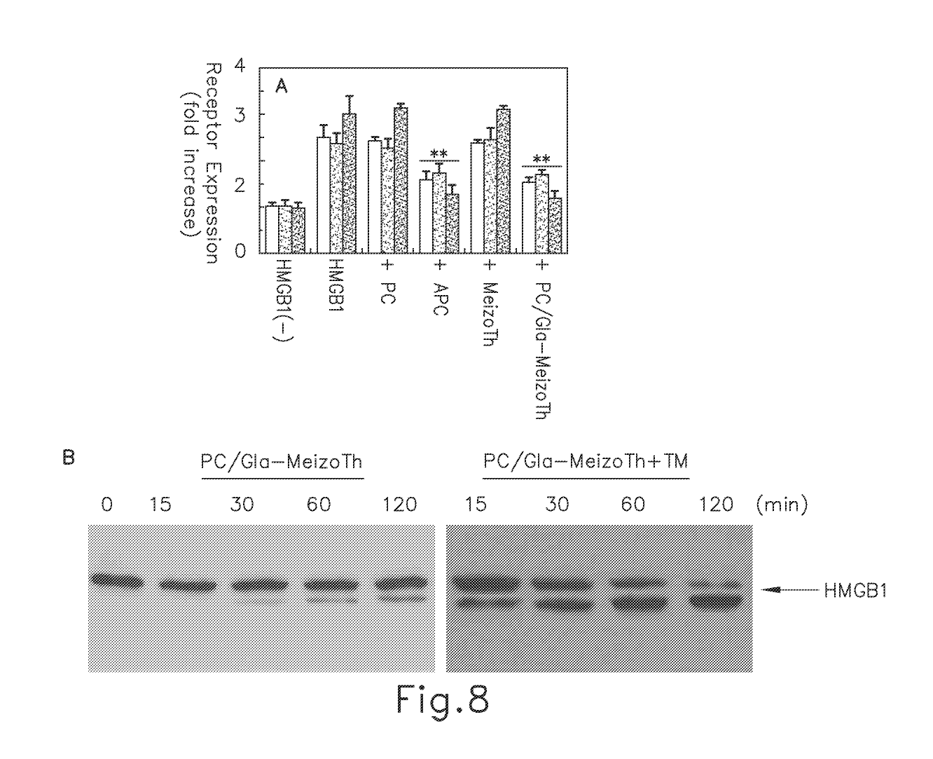

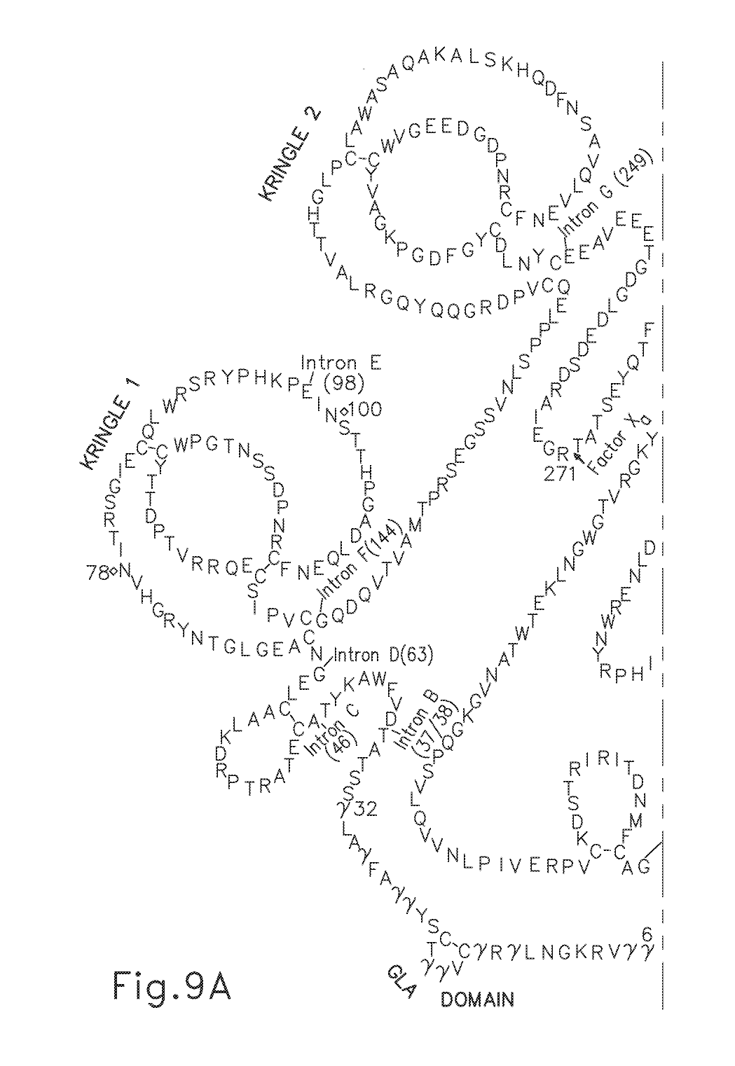

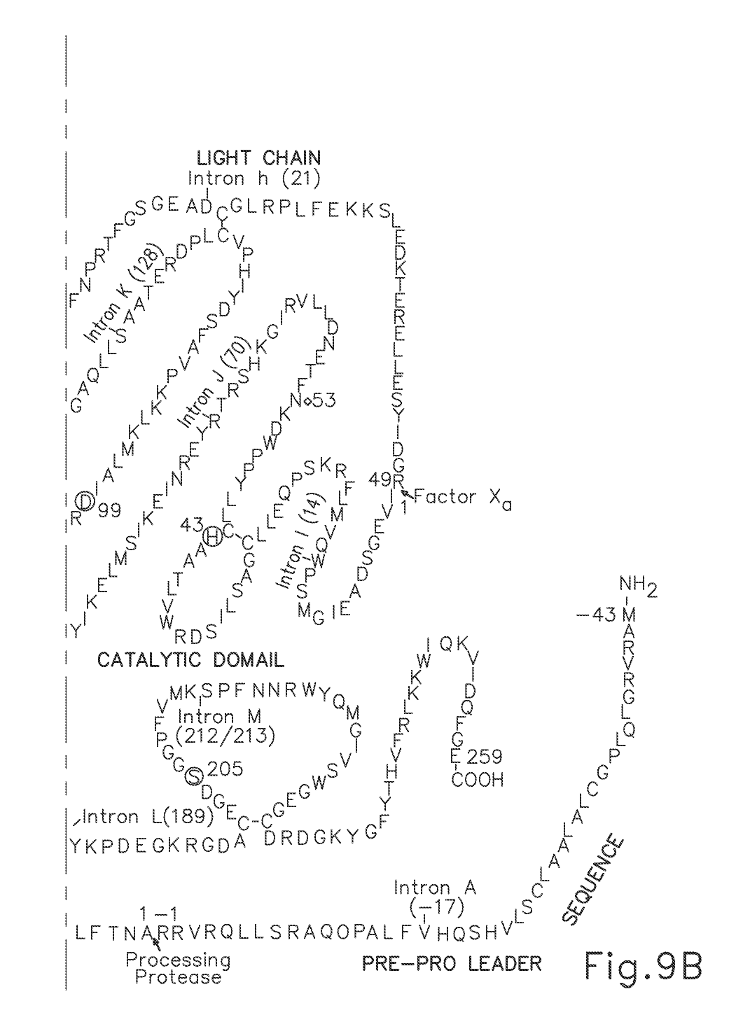

The effect of HMGB1 on the stimulation of its own receptors and the effect of APC on modulation of the expression of these receptors in endothelial cells was next investigated. As presented in FIG. 8A, HMGB1 induced the expression of all three receptors TLR2, TLR4 and RAGE by endothelial cells by about 2.5-3-fold. Interestingly, APC significantly inhibited the stimulatory effect of HMGB1 on all three receptors (FIG. 8A). The same results were observed with PCgla/MeizoTh except that a markedly lower concentration of the mutant protease was required to obtain a similar inhibitory effect on the expression of the receptors (FIG. 8A). The stimulatory effect of HMGB1 on the expression of RAGE was higher than the other two receptors and the inhibitory effect of the proteases (APC and PCgla/MeizoTh) on the expression of this receptor was also more pronounced.

PCgla/MeizoTh in Complex with TM Cleaves HMGB1

Previous results have indicated that thrombin in complex with TM can inhibit the proinflammatory effect of HMGB1 by cleaving the protein. [25] Since the TM-binding exosite-1 of thrombin is also expressed on meizothrombin, [30] it was decided to investigate whether PCgla/MeizoTh can cleave HMGB1 in the presence of TM. The results presented in FIG. 8B indicated that PCgla/MeizoTh can proteolytically cleave HMGB1 and that TM can function as a cofactor to markedly accelerate the cleavage reaction. By contrast, APC had no direct proteolytic effect on HMGB1 since its incubation with 100 nM APC in the absence and presence of soluble EPCR and/or PC/PS/PE vesicles did not lead to the degradation of HMGB1 even after 2 hours incubation at 37° C. (data not shown).

### DISCUSSION

In this study, it was demonstrated for the first time that APC inhibits the LPS-mediated secretion of HMGB1 by endothelial cells as well as the HMGB1-mediated pro-inflammatory signaling responses in endothelial cells by EPCR and PAR-1 dependent mechanisms. The optimal inhibitory activity of APC in down-regulating the HMGB1 secretion and signaling was observed at an APC concentration of 100 nM. APC down-regulated the HMGB1-mediated expression of the cell surface endothelial cell adhesion molecules, ICAM-1, VCAM-1 and E-selectin, thereby inhibiting both the interaction of the monocytic THP-1 cells with the activated endothelial cells and their subsequent migration across the monolayer. The in vitro antiinflammatory effects of APC were mediated through its inhibiting the HMGB1-mediated activation of the NF-κB pathway as well as suppressing the induction of TNF-α in endothelial cells in response to HMGB1. APC not only down-regulated the expression of HMGB1 and its proinflammatory signaling through the inhibition of the NF-κB pathway, but it also down-regulated the cell surface expression of the three receptors, TLR2, TLR4 and RAGE which are known to bind HMGB1 to initiate pro-inflammatory responses in endothelial cells. [6-8]

In view of the discovery that the Gla-domain of APC is responsible for its protective anti-inflammatory activity, it was hypothesized that, if this domain was switched with the Gla-domain of a prothrombin derivative that on being activated by Factor Xa produces the active intermediate meizothrombin (FIG. 1), this would yield a meizothrombin derivative (PCgla/MeizoTh) that would bind to EPCR with similar affinity as APC and activate PAR-1 with much higher efficacy similar to that observed with thrombin, and therefore this molecule may act as a potent anti-inflammatory molecule. This was indeed the case, as PCgla/MeizoTh was found to be characterized by all the anti-inflammatory activities of APC but with a 20 to 50-fold higher efficacy than APC.

In support of the three receptors, TLR2, TLR4 and RAGE mediating the intracellular signaling activities of HMGB1, the specific siRNA for each receptor significantly inhibited the signaling function of HMGB1. The simultaneous transfection of endothelial cells with siRNA for all three receptors resulted in an additive inhibitory effect, suggesting that all three receptors contribute to HMGB1 signaling, though the specific siRNA for RAGE appeared to be the most effective inhibitor of HMGB1 signaling in endothelial cells. APC also inhibited the expression of RAGE more effectively than the expression of the other two receptors.

Activation of the endothelium by pro-inflammatory cytokines during infection plays an important pathophysiological role in the chain of events that may lead to the septic shock/severe sepsis syndrome. The results presented above, together with those previously reported by others, [9-11] suggest that vascular endothelial cells may be rich sources of HMGB1 which can be released in response to bacterial endotoxin and endogenously expressed pro-inflammatory cytokines, thereby contributing to the pathology of severe sepsis. In support of HMGB1 playing an important pathological role in severe sepsis, it has been found that high plasma levels of HMGB1 in patients with severe sepsis and in animal models of endotoxemia correlate with higher mortality. [5, 12] Moreover, the administration of exogenous HMGB1 to experimental animals has lead to an elaboration of severe inflammatory responses, tissue injury and death. [1, 5, 11]

Further support of a deleterious effect of HMGB1 in severe sepsis is provided by the observations that neutralizing antibodies, chemical inhibitors and antagonists of HMGB1 release have all protected animals from the lethality of endotoxemia. [5] Thus, the finding that APC inhibits the secretion of HMGB1 and its proinflammatory signaling function through the three receptors TLR2, TLR4 and RAGE strongly suggests that the APC inhibition of this late acting inflammatory mediator may contribute to its mortality reducing protective activity against severe sepsis.

It was also found that, unlike meizothrombin which up-regulated the HMGB1 signaling pathway, the chimeric meizothrombin mutant containing the Gla-domain of protein C (PCgla/MeizoTh) inhibited the expression of HMGB1 and its signaling function through the same three cell surface receptors with about 20 to 50-fold higher efficacy than APC. Thus, when EPCR is occupied by the Gla-domain of protein C, the activation of PAR-1 by coagulation proteases initiates protective responses in endothelial cells exposed to pro-inflammatory mediators. [20,26]

Because of its about 20 to 50-fold improved efficacy, PCgla/MeizoTh can have therapeutic superiority over APC in treating patients with infection-induced inflammatory responses such as sepsis. In this context, it is also of important to note that PCgla/MeizoTh has minimal procoagulant activity since, unlike thrombin, it cannot effectively cleave fibrinogen, but, similar to thrombin, it can bind to TM to rapidly catalyze the activation of protein C to APC. [30] Thus, a further advantage of PCgla/MeizoTh as a potential therapeutic molecule is that it can activate protein C when it binds to TM, thereby amplifying the antiinflammatory responses through the APC pathway. Moreover, similar to the thrombin-TM complex, [25] PCgla/MeizoTh in complex with TM can cleave HMGB1 to down-regulate its pro-inflammatory signaling activities by a proteolytic pathway (as shown in FIG. 8B). This is in agreement with previous results showing that the thrombin-cleaved HMGB1 has significantly decreased pro-inflammatory properties. [25]

In view of the above, it will be seen that several advantages of the invention are achieved and other advantageous results attained.

Not all of the depicted components illustrated or described may be required. In addition, some implementations and embodiments may include additional components. Variations in the arrangement and type of the components may be made without departing from the spirit or scope of the claims as set forth herein. Additional, different or fewer components may be provided and components may be combined. Alternatively or in addition, a component may be implemented by several components.

The above description illustrates the invention by way of example and not by way of limitation. This description clearly enables one skilled in the art to make and use the invention, and describes several embodiments, adaptations, variations, alternatives and uses of the invention, including what is presently believed to be the best mode of carrying out the invention. Additionally, it is to be understood that the invention is not limited in its application to the details of construction and the arrangement of components set forth in the following description or illustrated in the drawings. The invention is capable of other embodiments and of being practiced or carried out in various ways. Also, it will be understood that the phraseology and terminology used herein is for the purpose of description and should not be regarded as limiting.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

### REFERENCES

1. Wang H, Bloom O, Zhang M, et al. HMG-1 as a late mediator of endotoxin lethality in mice. Science. 1999; 285(5425): 248-251.
2. Scaffidi P, Misteli T, Bianchi M E. Release of chromatin protein HMGB1 by necrotic cells triggers inflammation. Nature. 2002; 418(6894): 191-195.
3. DeMarco R A, Fink M P, Lotze M T. Monocytes promote natural killer cell interferon gamma production in response to the endogenous danger signal HMGB1. Mol. Immunol. 2005; 42(4): 433-444.
4. Andersson U, Wang H, Palmblad K, et al. High mobility group 1 protein (HMG-1) stimulates proinflammatory cytokine synthesis in human monocytes. J Exp Med. 2000; 192(4): 565-570.
5. Sama A E, D'Amore J, Ward M F, Chen G, Wang H. Bench to bedside:
HMGB1-a novel proinflammatory cytokine and potential therapeutic target for septic patients in the emergency department. Acad Emerg Med. 2004; 11(8):867-873.
6. Park J S, Svetkauskaite D, He Q, Kim J Y, Strassheim D, Ishizaka A, Abraham E. Involvement of toll-like receptors 2 and 4 in cellular activation by high mobility group box 1 protein. J Biol. Chem. 2004; 279(9): 7370-7377.
7. Hori O, Brett J, Slattery T, et al. The receptor for advanced glycation end products (RAGE) is a cellular binding site for amphoterin. Mediation of neurite outgrowth and co-expression of rage and amphoterin in the developing nervous system. J Biol. Chem. 1995; 270(43): 25752-25761.
8. Lotze M T, Tracey K J. High-mobility group box 1 protein (HMGB1): nuclear weapon in the immune arsenal. Nat Rev Immunol. 2005; 5(4): 331-342.
9. Fiuza C, Bustin M, Talwar S, Tropea M, Gerstenberger E, Shelhamer J H, Suffredini A F. Inflammation-promoting activity of HMGB1 on human microvascular endothelial cells. Blood. 2003; 101(7): 2652-2660.
10. Treutiger C J, Mullins G E, Johansson A S, et al. High mobility group 1 B-box mediates activation of human endothelium. J Intern Med. 2003; 254(4): 375-85.
11. Mullins G E, Sunden-Cullberg J, Johansson A S, et al. Activation of human umbilical vein endothelial cells leads to relocation and release of high-mobility group box chromosomal protein 1. Scand J. Immunol. 2004; 60(6):566-573.
12. Chen G, Ward M F, Sama A E, Wang H. Extracellular HMGB1 as a proinflammatory cytokine. J Interferon Cytokine Res. 2004; 24(6): 329-333.
13. Bernard G R, Vincent J L, Laterre P F, et al. Recombinant human protein C Worldwide Evaluation in Severe Sepsis (PROWESS) study group. Efficacy and safety of recombinant human activated protein C for severe sepsis. N Engl J. Med. 2001; 344(10): 699-709.
14. Taylor F B, Stearns-Kurosawa D J, Kurosawa S, et al. The endothelial cell protein C receptor aids in host defense against Escherichia coli sepsis. Blood. 2000; 95(5): 1680-1686.
15. Riewald M, Petrovan R J, Donner A, Mueller B M, Ruf W. Activation of endothelial cell protease activated receptor 1 by the protein C pathway. Science. 2002; 296(5574):1880-1882.
16. Mosnier L O, Zlokovic B V, Griffin J H. The cytoprotective protein C pathway. Blood. 2007; 109(8): 3161-3172.
17. Kerschen E J, Fernandez J A, Cooley B C, et al. Endotoxemia and sepsis mortality reduction by non-anticoagulant activated protein C. J Exp Med. 2007; 204(10): 2439-2448.
18. Niessen, F.; Schaffner, F.; Furlan-Freguia, C.; Pawlinski, R.; Bhattacharjee, G.; Chun, J.; Derian, C. K.; Andrade-Gordon, P.; Rosen, H.; Ruf, W. Dendritic cell PAR1-S1 P3 signaling couples coagulation and inflammation. Nature, 2008 452, 654-658.
19. Joyce D E, Nelson D R, Grinnell B W. Leukocyte and endothelial cell interactions in sepsis: relevance of the protein C pathway. Crit. Care Med. 2004; 32(5 Suppl): S280-286.
20. Rezaie A R. Regulation of the protein C anticoagulant and antiinflammatory pathways. Curr Med. Chem. 2010; 17(19): 2059-2069.
21. Finigan J H, Dudek S M, Singleton P A, et al. Activated protein C mediates novel lung endothelial barrier enhancement: role of sphingosine 1-phosphate receptor transactivation. J Biol. Chem. 2005; 280(17): 17286-17293.
22. Feistritzer C, Riewald M. Endothelial barrier protection by activated protein C through PAR1-dependent sphingosine 1-phosphate receptor-1 crossactivation. Blood. 2005; 105(8): 3178-3184.
23. Zlokovic B V, Griffin J H. Cytoprotective protein C pathways and implications for stroke and neurological disorders. Trends Neurosci. 2011; 34(4): 198-209.
24. Xu J, Zhang X, Pelayo R, et al. Extracellular histones are major mediators of death in sepsis. Nat. Med. 2009; 15(11): 1318-1321.
25. Ito T, Kawahara K, Okamoto K, et al. Proteolytic cleavage of high mobility group box 1 protein by thrombin-thrombomodulin complexes. Arterioscler Thromb Vasc Biol. 2008; 28(10): 1825-1830.
26. Bae J S, Yang L, Manithody C, Rezaie A R. The ligand occupancy of endothelial protein C receptor switches the protease-activated receptor 1-dependent signaling specificity of thrombin from a permeability-enhancing to a barrier-protective response in endothelial cells. Blood. 2007; 110(12): 3909-3916.
27. Bae J S, Rezaie A R. Protease activated receptor 1 (PAR-1) activation by thrombin is protective in human pulmonary artery endothelial cells if endothelial protein C receptor is occupied by its natural ligand. Thromb Haemost. 2008; 100(1):101-109.
28. Kim I, Moon S O, Kim S H, Kim H J, Koh Y S, Koh G Y. Vascular endothelial growth factor expression of intercellular adhesion molecule 1 (ICAM-1), vascular cell adhesion molecule 1 (VCAM-1), and E-selectin through nuclear factor-kappa B activation in endothelial cells. J Biol. Chem. 2001; 276(10): 7614-7620.
29. Bae J S, Yang L, Manithody C, Rezaie A R. Engineering a disulfide bond to stabilize the calcium-binding loop of activated protein C eliminates its anticoagulant but not its protective signaling properties. J Biol. Chem. 2007; 282(12):9251-9259.
30. Côté H C, Bajzar L, Stevens W K, Samis J A, Morser J, MacGillivray R T, Nesheim M E. Functional characterization of recombinant human meizothrombin and Meizothrombin (desF1). Thrombomodulin-dependent activation of protein C and thrombin-activatable fibrinolysis inhibitor (TAFI), platelet aggregation, antithrombin-III inhibition. J Biol. Chem. 1997; 272(10):6194-6200.
31. Bae J S, Rezaie A R. Activated protein C inhibits high mobility group box 1 signaling in endothelial cells. Blood, 2011.
32. A: Esmon, C. T. Molecular events that control the protein C anticoagulant pathway. Thromb. Haemost., 1993, 70, 29-35.
33. Griffin, J. H.; Evatt, B.; Zimmerman, T. S.; Kleiss, A. J.; Wideman, C. Deficiency of protein C in congenital thrombotic disease. J. Clin. Invest., 1981, 68, 1370-1373.
34. Bae J S, Kim Y U, Park M K, Rezaie A R. Concentration dependent dual effect of thrombin in endothelial cells via Par-1 and Pi3 Kinase. J Cell Physiol 2009 June; 219(3): 744-751.
35. Lisa M. Regan, Jeffery S. Mollica, Alireza R. Rezaie, and Charles T. Esmon. The interaction between the endothelial cell Protein C receptor and Protein C is dictated by the g-arboxyglutamic acid domain of Protein C. J Biol Chem 1997 October; 272 (42): 26279-26284.

1. A method for inhibiting the release of HMGB1 from a cell of an individual, comprising administering to said individual an effective amount of a composition comprising a chimeric protein, the chimeric protein comprising: (a) a prothrombin sequence, and (b) a PC sequence, wherein the chimeric protein has substantially no procoagulant activity.

2. The method of claim 1, wherein the chimeric protein comprises a prothrombin proteinase domain, a prothrombin Kringle-1 domain, and a prothrombin Kringle-2 domain.

3. The method of claim 1, wherein the chimeric protein comprises a PC Gla-domain.

4. The method of claim 1, wherein the prothrombin sequence comprise the amino acid sequence of SEQ ID NO: 3.

5. The method of claim 1, wherein the PC sequence comprises the amino acid sequence of SEQ ID: NO. 4.

6. The method of claim 1, wherein the chimeric protein comprises the sequence of SEQ ID NO:5.

7. The method of claim 1, wherein the chimeric protein comprises an amino acid replacement of arginine with an amino acid other than arginine at a locus corresponding to amino acid arginine-271 of a thrombin polypeptide comprising an amino acid sequence set forth in SEQ ID NO:1.

8. The method of claim 7, wherein the amino acid residue replacing said arginine is selected from the group consisting of Ala, Val, Leu, Ile, Met, Gln, Glu, Gly, His, Met, Ser, Thr, Trp, and Tyr.

9. The method of claim 8, wherein the amino acid residue replacing said arginine is an alanine residue.

10. The method of claim 1, wherein the chimeric protein comprises an amino acid replacement of arginine with an amino acid other than arginine at a locus corresponding to amino acid arginine-155 of a thrombin polypeptide comprising an amino acid sequence set forth in SEQ ID NO:1.

11. The method of claim 10, wherein the amino acid residue replacing said arginine is an alanine residue.

12. The method of claim 1, wherein the chimeric protein comprises an amino acid replacement of arginine with an amino acid other than arginine at a locus corresponding to amino acid arginine-284 of a thrombin polypeptide comprising an amino acid sequence set forth in SEQ ID NO:1.

13. A composition for inhibiting the release of HMGB1 from a cell, comprising a chimeric protein, the chimeric protein comprising: (a) a prothrombin sequence, and (b) a PC sequence, wherein the chimeric protein has substantially no procoagulant activity.

14. The composition of claim 13, wherein the chimeric protein comprises a prothrombin proteinase domain, a prothrombin Kringle-1 domain, and a prothrombin Kringle-2 domain.

15. The composition of claim 13, wherein the chimeric protein comprises a PC Gla-domain.

16. The composition of claim 13, wherein the chimeric protein comprises the sequence of SEQ ID NO:5.

17. A composition for reducing the amount of HMGB1 in plasma, comprising a chimeric protein, the chimeric protein comprising: (a) a prothrombin sequence, and (b) a PC sequence, wherein the chimeric protein has substantially no procoagulant activity.

18. The composition of claim 17, wherein the chimeric protein comprises a prothrombin proteinase domain, a prothrombin Kringle-1 domain, and a prothrombin Kringle-2 domain.

19. The composition of claim 17, wherein the chimeric protein comprises a PC Gla-domain.

20. The composition of claim 17, wherein the chimeric protein comprises the sequence of SEQ ID NO:5.


SEQUENCE LISTING (5 sequences)

SEQ ID NO: 1 - DNA, 1920 bp, Homo sapiens
  exon (1)..(1869)
  misc_feature (198)..(198): Residue “Arg” in position 198 is a mutation
atggcccgcatccgaggcttgcagctgcctggctgcctggccctggctgccctgtgtagccttgtgcacagccagcatgtgttcctggctcctcagcaagcacggtcgctgctccagcgggtccggcgagccaacaccttcttggaggaggtgcgcaagggcaacctagagcgagagtgcgtggaggagacgtgcagctacgaggaggccttcgaggctctggagtcctccacggctacggatgtgttctgggccaagtacacagcttgtgagacagcgaggacgcctcgagataagcttgctgcatgtctggaaggtaactgtgctgagggtctgggtacgaactaccgagggcatgtgaacatcacccggtcaggcattgagtgccagctatggaggagtcgctacccacataagcctgaaatcaactccactacccatcctggggccgacctacaggagaatttctgccgcaaccccgacagcagcaacacgggaccctggtgctacactacagaccccaccgtgaggaggcaggaatgcagcatccctgtctgtggccaggatcaagtcactgtagcgatgactccacgctccgaaggctccagtgtgaatctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgtgtggtgctatgtggccgggaagcctggcgactttgggtactgcgacctcaactattgtgaggaggccgtggaggaggagacaggagatgggctggatgaggactcagacagggccatcgaagggcgtaccgccacaagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagggggccactcatattctgggctcctggaaccaatcccgtgaaagaattatt

SEQ ID NO: 2 - DNA, 1500 bp, Homo sapiens
  exon (74)..(1456)
gctgtcatggcggcaggacggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggat

SEQ ID NO: 3 - DNA, 1656 bp, Homo sapiens
  exon (1)..(1602)
  misc_feature (110)..(110): Residue “Arg” in position 110 is a mutation
gcttgtgagacagcgaggacgcctcgagataagcttgctgcatgtctggaaggtaactgtgctgagggtctgggtacgaactaccgagggcatgtgaacatcacccggtcaggcattgagtgccagctatggaggagtcgctacccacataagcctgaaatcaactccactacccatcctggggccgacctacaggagaatttctgccgcaaccccgacagcagcaacacgggaccctggtgctacactacagaccccaccgtgaggaggcaggaatgcagcatccctgtctgtggccaggatcaagtcactgtagcgatgactccacgctccgaaggctccagtgtgaatctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgtgtggtgctatgtggccgggaagcctggcgactttgggtactgcgacctcaactattgtgaggaggccgtggaggaggagacaggagatgggctggatgaggactcagacagggccatcgaagggcgtaccgccacaagtgagtaccagactttcttcaatccgaggacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagggggccactcatattctgggctcctggaaccaatcccgtgaaagaattatt

SEQ ID NO: 4 - DNA, 261 bp, Homo sapiens
  exon (1)..(261)
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtc

SEQ ID NO: 5 - DNA, 1917 bp, Homo sapiens
  exon (1)..(1863)
  misc_feature (197)..(197): Residue “Ala” in position 197 is a mutation
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgcttgtgagacagcgaggacgcctcgagataagcttgctgcatgtctggaaggtaactgtgctgagggtctgggtacgaactaccgagggcatgtgaacatcacccggtcaggcattgagtgccagctatggaggagtcgctacccacataagcctgaaatcaactccactacccatcctggggccgacctacaggagaatttctgccgcaaccccgacagcagcaacacgggaccctggtgctacactacagaccccaccgtgaggaggcaggaatgcagcatccctgtctgtggccaggatcaagtcactgtagcgatgactccagcttccgaaggctccagtgtgaatctgtcacctccattggagcagtgtgtccctgatcgggggcagcagtaccaggggcgcctggcggtgaccacacatgggctcccctgcctggcctgggccagcgcacaggccaaggccctgagcaagcaccaggacttcaactcagctgtgcagctggtggagaacttctgccgcaacccagacggggatgaggagggcgtgtggtgctatgtggccgggaagcctggcgactttgggtactgcgacctcaactattgtgaggaggccgtggaggaggagacaggagatgggctggatgaggactcagacagggccatcgaaggggctaccgccacaagtgagtaccagactttcttcaatccggctacctttggctcgggagaggcagactgtgggctgcgacctctgttcgagaagaagtcgctggaggacaaaaccgaaagagagctcctggaatcctacatcgacgggcgcattgtggagggctcggatgcagagatcggcatgtcaccttggcaggtgatgcttttccggaagagtccccaggagctgctgtgtggggccagcctcatcagtgaccgctgggtcctcaccgccgcccactgcctcctgtacccgccctgggacaagaacttcaccgagaatgaccttctggtgcgcattggcaagcactcccgcaccaggtacgagcgaaacattgaaaagatatccatgttggaaaagatctacatccaccccaggtacaactggcgggagaacctggaccgggacattgccctgatgaagctgaagaagcctgttgccttcagtgactacattcaccctgtgtgtctgcccgacagggagacggcagccagcttgctccaggctggatacaaggggcgggtgacaggctggggcaacctgaaggagacgtggacagccaacgttggtaaggggcagcccagtgtcctgcaggtggtgaacctgcccattgtggagcggccggtctgcaaggactccacccggatccgcatcactgacaacatgttctgtgctggttacaagcctgatgaagggaaacgaggggatgcctgtgaaggtgacagtgggggaccctttgtcatgaagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagggggccactcatattctgggctcctggaaccaatcccgtgaaagaattatt